(19) United States
(12) Patent Application Publication
(10) Pub. No.: US 2023/0335293 A1
(43) Pub. Date: Oct. 19, 2023

(54) METHODS AND SYSTEMS FOR ACCELERATING QUANTITATIVE SYSTEMS PHARMACOLOGY (QSP) MODELS

(71) Applicant: JuliaHub, Inc., Boston, MA (US)

(72) Inventors: Christopher RACKAUCKAS, Cambridge, MA (US); Viral B. SHAH, Cambridge, MA (US); Sebastian Miclu¿a-Câmpeanu, Bucharest (RO)

(21) Appl. No.: 18/136,654

(22) Filed: Apr. 19, 2023

Related U.S. Application Data
(60) Provisional application No. 63/332,589, filed on Apr. 19, 2022.

(51) Int. Cl. G16H 50/50; G06T 17/00; G06F 17/13; G06F 17/18

(52) U.S. Cl. CPC G16H 50/50; G06T 17/00; G06F 17/13; G06F 17/18

(57) ABSTRACT

Systems and methods for design optimization using a multiple-data fitting interface are disclosed. Data objects that include pairs of data with information in the form of one or more computational models that model the behavior of multiple items are received. The received data objects are paired into a collection that includes dependencies between the data objects. A virtual population is generated based on the received data objects. The virtual population comprises multiple virtual patients, with each virtual patient comprising a combination of model parameters that describe data in the received data objects. A prediction for the virtual population is generated by simulating with each of the virtual patients. The generation of the virtual population includes a user-determined or default cost function and an algorithm for finding an optimal configuration for the virtual population with respect to said cost. An output is generated that includes a visualization of the prediction.

### GOVERNMENT CROSS-REFERENCE TO RELATED APPLICATIONS

This application claims priority to U.S. Provisional Patent Application No. 63/332,589 filed on Apr. 19, 2022 by JuliaHub, Inc. entitled “METHODS AND SYSTEMS FOR ACCELERATING QUANTITATIVE SYSTEMS PHARMACOLOGY (QSP) MODELS”, the entire content of which is incorporated by reference herein.

### GOVERNMENT SUPPORT

This invention was made with U.S. Government support by National Science Foundation under NSF award IIP-1938400. The Government has certain rights in this invention.

### TECHNICAL FIELD

The field of the invention relates generally to methods and systems for quantitative systems pharmacology (QSP). More specifically, invention relates to accelerating QSP models by training machine learning (ML) approximations on simulations.

### BACKGROUND

Quantitative systems pharmacology (QSP) refers to the quantitative analysis of the dynamic interactions between drug(s) and a biological system that aims to understand the behavior of the system as a whole. QSP is an approach to model-informed drug discovery and development (MIDD) that can be used to generate biological/pharmacological hypotheses in silico to aid in the design of in vitro or in vivo non-clinical and clinical experiments. QSP is also a computational model that examines the interface between discrete experimental data any biological process such as physiological consequences of a disease, a specific disease pathway, or genomics, proteomics, metabolomics, etc. QSP can be employed at all stages of drug development.

In contrast to traditional empirical or mechanistic pharmacokinetic (PK)/pharmacodynamic (PD) models designed to characterize one or more specific, but similar, datasets to generate inferences and predict results for related scenarios, QSP models investigate the effects of drug action based on emergent behaviors of the underlying system. To do so, QSP models integrate datasets from diverse contexts into a mathematical framework that reflects knowledge of the system, and these QSP models can then predict outcomes in untested scenarios.

One problem with QSP model development, however, is that collecting enough clinical trial data is often infeasible or impossible, which creates an imbalance between model complexity and available data. This imbalance between model complexity and available data can create issues. For example, when model parameters are learned from data, it is often the case that many different parameters can describe given data equally or sufficiently well. This results in a high uncertainty in the parameter estimation.

Another problem is that QSP is often hindered by its computational complexity. As a result, faster compute speeds are often required for faster drug development because the compute time required to perform simulations affects the speed at which researchers can iterate through model development and perform analyses such as global sensitivity analysis. Given that these aspects are some of the most time-consuming aspects of the preclinical analysis pipeline, it is desirable to accelerate these computations.

Yet another problem associated with current QSP models is their tendency to capture multiple timescale phenomena. For example, model features such as circadian rhythm or metabolite digestion can span hours while the individual dynamics of important transcription factors can act in microseconds. This timescale separation leads to numerical issues which manifest in highly ill-conditioned Jacobians, a phenomenon known as “stiffness” in the field of ordinary differential equations (ODEs). In QSP, this issue of capturing multiple timescale phenomena often manifests itself as instability of explicit method ODE solvers, such as MATLAB's ode45, in the presence of these effects.

Accordingly, a need exists for improved methods and systems for accelerating QSP modeling while providing configurable and accurate results.

### SUMMARY

This summary is provided to introduce a selection of concepts in a simplified form that are further described below in the detailed description. This summary is not intended to identify key features or essential features of the claimed subject matter, nor is it intended to be used to limit the scope of the claimed subject matter.

As explained in more detail below, the systems and methods disclosed herein provide for an improvement in computing technology because they allow for acceleration of QSP models that would otherwise be too resource-intensive and/or complex to be performed in a meaningful way. These improvements allow for new data objects and models to be created to simulate virtual clinical trials for investigation without the expense and time required to undertake a physical clinical trial, and they provide additional avenues for generating clinical trial data that would otherwise be infeasible or impossible to capture. They further allow for expansion of analyses of a clinical trial to dimensions where, for example, experimental data collection is difficult, by scaling known data from a clinical scoping and expanding that data to a virtual scope using virtual patients.

Systems and methods for design optimization using a multiple-data fitting interface are disclosed. Data objects that include pairs of data with information in the form of one or more computational models that model the behavior of multiple items in a system are received. The received data objects are paired into a collection that includes dependencies between the received data objects. A virtual population is generated based on the received data objects. The virtual population comprises multiple virtual patients, with each virtual patient comprising a combination of model parameters that describe data in the received data objects. A prediction for the virtual population is generated by simulating with each of the virtual patients. The generation of the virtual population includes a user-determined or default cost function and an algorithm for finding an optimal configuration for the virtual population with respect to said cost. An output is generated that includes a visualization of the prediction for the virtual population.

In an embodiment disclosed herein, a system having a multiple-data fitting interface for design optimization is disclosed. The system includes at least one processor configured for receiving data objects that include pairs of data with information in the form of one or more computational models that model the behavior of multiple items in a system. The at least one processor is further configured for pairing the received data objects into a collection. The collection includes dependencies between the received data objects. The at least one processor is further configured for generating a virtual population based on the received data objects. The virtual population includes multiple virtual items, with each virtual item including a combination of model parameters that describe data in the received data objects. The at least one processor is further configured for generating a prediction for the virtual population using a global sensitivity analysis across the virtual population. The prediction includes a determined cost and an optimal configuration for the virtual population. The at least one processor is further configured for generating an output. The output includes a visualization of the prediction for the virtual population.

In another embodiment disclosed herein, a method for design optimization using a multiple-data fitting interface is disclosed. The method includes receiving data objects that include pairs of data with information in the form of one or more computational models that model the behavior of multiple items in a system. The method further includes pairing the received data objects into a collection. The collection includes dependencies between the received data objects. The method further includes generating a virtual population based on the received data objects. The virtual population comprises multiple virtual items, with each virtual item comprising a combination of model parameters that describe data in the received data objects. The method further includes generating a prediction for the virtual population using a global sensitivity analysis across the virtual population. The prediction includes a determined cost and an optimal configuration for the virtual population. The method further includes generating an output, wherein the output includes a visualization of the prediction for the virtual population.

In another embodiment disclosed herein, a system having a multiple-data fitting interface for design optimization is disclosed. The system includes at least one processor configured for receiving data objects that include pairs of data with information in the form of one or more computational models that model the behavior of multiple patients in a clinical trial. The at least one processor is further configured for pairing the received data objects into a collection that includes dependencies between the received data objects. The at least one processor is further configured for generating a virtual population based on the received data objects. The virtual population comprises multiple virtual patients, with each virtual patient including a combination of model parameters that describe data in the received data objects. The generation of the virtual population includes a user-determined or default cost function and an algorithm for finding an optimal configuration for the virtual population with respect to said cost. The at least one processor is further configured for generating a prediction for the virtual population by simulating with each of the virtual patients. The at least one processor is further configured for generating an output. The output includes a visualization of the prediction for the virtual population.

In another embodiment disclosed herein, a method for design optimization using a multiple-data fitting interface is disclosed. The method includes receiving data objects that include pairs of data with information in the form of one or more computational models that model the behavior of multiple patients in a clinical trial. The method further includes pairing the received data objects into a collection that includes dependencies between the received data objects. The method further includes generating a virtual population based on the received data objects. The virtual population comprises multiple virtual patients, with each virtual patient including a combination of model parameters that describe data in the received data objects. The generation of the virtual population includes a user-determined or default cost function and an algorithm for finding an optimal configuration for the virtual population with respect to said cost. The method further includes generating a prediction for the virtual population by simulating with each of the virtual patients. The method further includes generating an output. The output includes a visualization of the prediction for the virtual population.

According to one system, at least one processor is configured for receiving data objects that include pairs of data with information in the form of one or more computational models that model the behavior of multiple items in a system. The at least one processor is further configured for pairing the received data objects into a collection that includes dependencies between the received data objects. The at least one processor is further configured for generating a virtual population based on the received data objects. The virtual population comprises multiple virtual patients, with each virtual patient comprising a combination of model parameters that describe data in the received data objects. The processor is further configured for generating a prediction for the virtual population by simulating with each of the virtual patients. The generation of the virtual population includes a user-determined or default cost function and an algorithm for finding an optimal configuration for the virtual population with respect to said cost. The processor is further configured for generating an output that includes a visualization of the prediction for the virtual population.

### BRIEF DESCRIPTION OF THE DRAWINGS

The present embodiments are illustrated by way of example and are not intended to be limited by the figures of the accompanying drawings.

FIG. 1 depicts a life cycle of a QSP model which may include defining a model, performing model simulations, analyzing the models, and reporting and/or interpreting the analysis.

FIG. 2 depicts stages of a process and their associated software in a Julia programming environment.

FIG. 3 depicts example source code for performing symbolic model definition using ModelingToolKit.jl's ODESystem.

FIG. 4 depicts a symbolic domain-specific language representation.

FIGS. 5A and 5B depict exemplary source code and software extension for loading or importing models according to an embodiment of the subject matter described herein.

FIG. 6 depicts exemplary computer-executable source code for Pumas-QSP IPSP and integration with Pumas according to an embodiment of the subject matter described herein.

FIGS. 7A and 7B depict various charts illustrating data, model simulation, and predictions for different trial types including a normal trial and a steady state trial according to an embodiment of the subject matter described herein.

FIGS. 8A and 8B depict various charts illustrating data, model simulation, and predictions for different trial types including a periodic steady state trial and a custom dosing trial according to an embodiment of the subject matter described herein.

FIGS. 9A and 9B depict a flow chart illustrating exemplary steps in a process for determining trial collections and types according to an embodiment of the subject matter described herein.

FIGS. 10A and 10B depict a flow chart illustrating an exemplary process for generating virtual populations.

FIGS. 11A and 11B depict a schematical overview of the optimization process at a per-trial level.

FIG. 12 depicts a schematical overview of receiving the virtual population generated as shown in FIGS. 11A and 11B.

FIG. 13 depicts a virtual trial, perturbing a value alpha and allowing the researchers to see the effect on unmeasured quantities.

FIGS. 14A and 14B showcase how the virtual trial returns a set of predictions for the perturbed trial for the set of patients.

FIG. 15 depicts exemplary source code and a virtual trial output.

FIG. 16 depicts additional exemplary source code and a virtual trial output.

FIG. 17 depicts the similar workflow for global sensitivity analysis (GSA), where a GSA method is computed on the outputs of the trials collected into the QSPSensitivity object.

FIGS. 18A-18D describe what global sensitivity analysis (GSA) methods measure.

FIGS. 19A and 19B depict an exemplary user interface of JuliaHub implementing Pumas-QSP according to an embodiment of the subject matter described herein.

FIG. 20 depicts a bar chart illustrating relative acceleration of a large simulation for a cardiac steady state calculation when using Pumas-QSP versus Matlab.

FIGS. 21A and 21B depict bar charts illustrating relative acceleration of a large simulation for a global optimization of a Leucine model and GPU acceleration when using Pumas-QSP versus Matlab+C+Sundials.

FIG. 22 depicts a line graph of the relationship between time and error for fast model simulations performed by DifferentialEquations.jl for exemplary Hires equations of nine stiff chemical reactions.

FIG. 23 depicts exemplary computer-executable source code for defining a QSP model with ModelingToolkit.jl.

FIG. 24 depicts exemplary computer-executable source code for reading and analyzing trial data according to an embodiment of the subject matter described herein.

FIG. 25 depicts a flow chart illustrating exemplary steps in a process for receiving user input according to an embodiment of the subject matter described herein.

FIGS. 26A-26C depict charts illustrating AI acceleration modeling of QSP models, including an exemplary Arabidopsis model, when using Pumas-QSP according to an embodiment of the subject matter described herein.

FIG. 27 depicts bar charts illustrating the relative speedup of an exemplary Arabidopsis model when using Pumas-QSP versus Matlab SBML Toolbox.

FIG. 28 depicts an exemplary dynamic and automatic report generated according to an embodiment of the subject matter described herein.

FIG. 29 depicts a functional relationship between various software modules of the subject matter described herein.

FIG. 30 depicts a block diagram illustrating one embodiment of a computing device that implements the methods and systems described herein.

### DETAILED DESCRIPTION

The following description and figures are illustrative and are not to be construed as limiting. Numerous specific details are described to provide a thorough understanding of the disclosure. In certain instances, however, well-known or conventional details are not described in order to avoid obscuring the description. References to “one embodiment” or “an embodiment” in the present disclosure may be (but are not necessarily) references to the same embodiment, and such references mean at least one of the embodiments.

An object of the subject matter described herein includes providing a design of a multi-data fitting interface for design optimization. This interface is applicable to many engineering scenarios, such as designing and fitting parameters for quantitative systems pharmacology (QSP) models, HVAC models, and to the calibration of lithium-ion battery models, for example.

In one exemplary implementation, an object of the subject matter described herein includes providing a framework for modeling pharmacological systems with high performance and high usability. High performance includes fast simulations, thorough analysis, and robust testing. High usability includes intuitively capturing models, exploring the complexity of systems, and automatic parallelization.

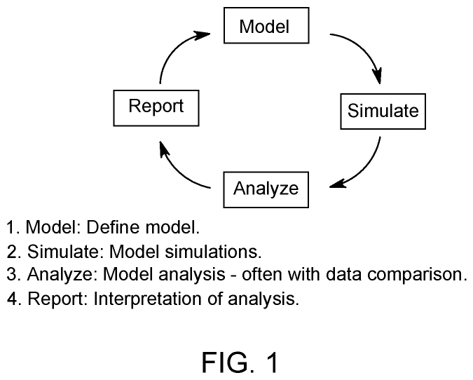

FIG. 1 depicts a life cycle of a QSP model, which may include defining a model, performing model simulations, analyzing the models, and reporting and/or interpreting the analysis. As will be discussed in greater detail below, a life cycle of a QSP model using Pumas-QSP may begin defining a QSP model intuitively. Next, models may be simulated efficiently. Pumas-QSP tools may then be used to analyze the model thoroughly. This includes creating diverse virtual populations based on large data sets (e.g., trial clinical data), exploring drug effects, and testing the drug effects. Lastly, a dynamic, automatic, and detailed report may be generated.

To address the shortcoming associated with current systems and methods mentioned above, a virtual population (VP) may be created and used in place of a real population, which provides significant savings in both cost and time to perform the clinical trial. As used herein, each combination of model parameter that describes data sufficiently well is considered a virtual patient. A collection of virtual patients is a VP. Virtual populations emulate the diversity in patients and can be use them to explore variability in effects in clinical trials. The information gathered from exploring the variability in effects in clinical trials can be used to craft more valuable physical clinical trials.

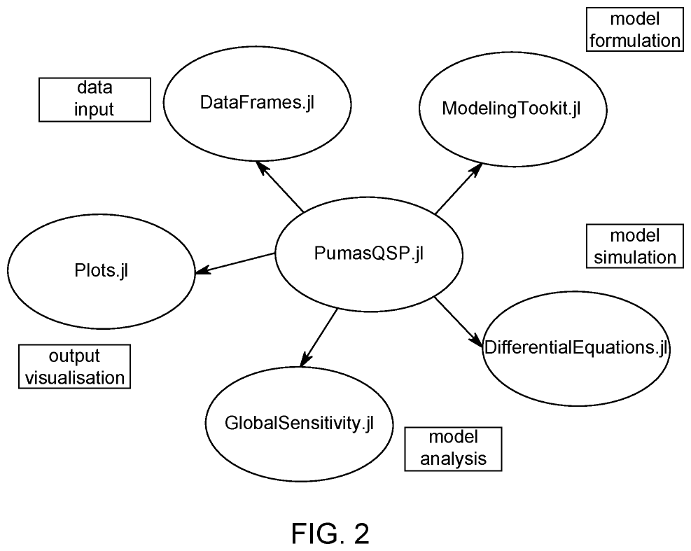

The subject matter described herein may be implemented via one or more software modules. The software modules may include non-transitory source code executed by a processor of a computing device. In one embodiment, the source code is written in the Julia programming language. As described in greater detail below with respect to FIG. 2, the methods disclosed herein may be implemented via a PumasQSP.jl module where PumasQSP.jl may be associated with one or more additional modules for implementing aspects or features herein.

PumasQSP.jl provides a method for creating virtual populations (VPs) and performing GSA in a high performance and user-friendly manner. One objective of the subject matter described herein includes allowing users to focus on the semantics of the model and the interpretation of its behavior. Clear and flexible code structures provide users with the opportunity to identify wider connections as well as narrow in on areas of interest. At the same time, automatic parallelizations enable users to formulate robust hypotheses and provide the necessary performance to allow users to test detailed hypotheses and explore options widely.

PumasQSP.jl, which is one possible implementation of the subject matter described herein, is written in the Julia programming language and therefore brings all the Julia language advantages with it. These advantages include, for example, an easy-to-read and -write syntax and a high baseline performance. Additionally, PumasQSP.jl is integrated in the existing package ecosystem of Julia. Connections between PumasQSP.jl and other software packages are described below.

FIG. 2 depicts stages of a process and their associated software in a Julia programming environment. According to one embodiment, data input is performed by DataFrames.jl. DataFrames.jl allows users to manipulate tabular data and hence is useful to handle data, such as clinical trial data, stored in, for example, csv files.

According to one embodiment, model formulation is performed by ModelingToolkit.jl. ModelingToolkit.jl provides a modeling framework for high-performance symbolic-numeric computation hence allows for smooth model definitions and model handling.

According to one embodiment, model simulation is performed by DifferentialEquations.jl. DifferentialEquations.jl can be used for numerically solving differential equations with high efficiency.

According to one embodiment, model analysis is performed by GlobalSensitivity.jl. GlobalSensitivity.jl provides several methods to quantify the uncertainty in the model output with respect to the input parameters of the model.

According to one embodiment, output visualization is performed by Plots.jl. Plots.jl is a plotting API and toolset that can be used to intuitively analyze and visualize model behavior and output.

The systems and methods described herein receive pairs of data (sometimes null data) with information about one or more computational models in objects, which are referred to herein as trials. The computational models may be, for example, some kind of differential equation, such as an ordinary differential equation (ODE), a partial differential equation (PDE), a stochastic differential equation (SDE), etc., or stochastic models such as those of Gillespie equations.

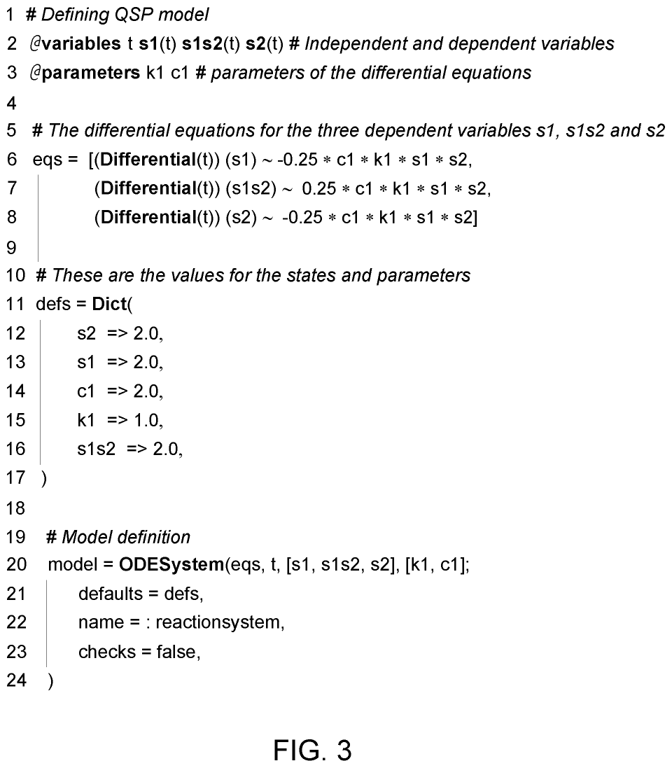

In some cases, these models may be provided symbolically and can be manipulated for further optimizations. FIG. 3, for example, depicts example source code for performing symbolic model definition using ModelingToolkit.jl's ODESystem.

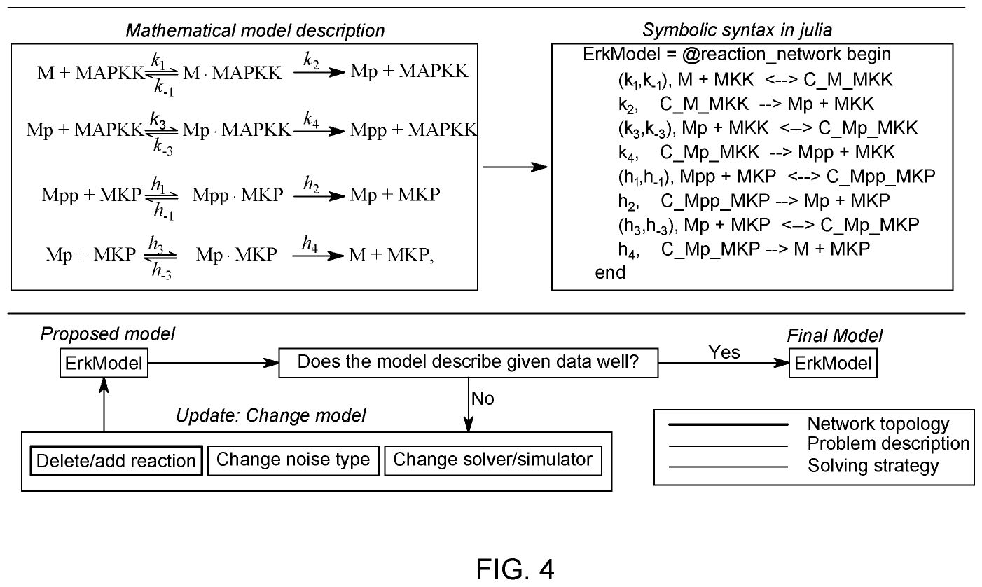

In some cases, these models may be defined using a graphical user interface (GUI), or symbolic domain-specific language representations. FIG. 4, for example, depicts a symbolic domain-specific language representation.

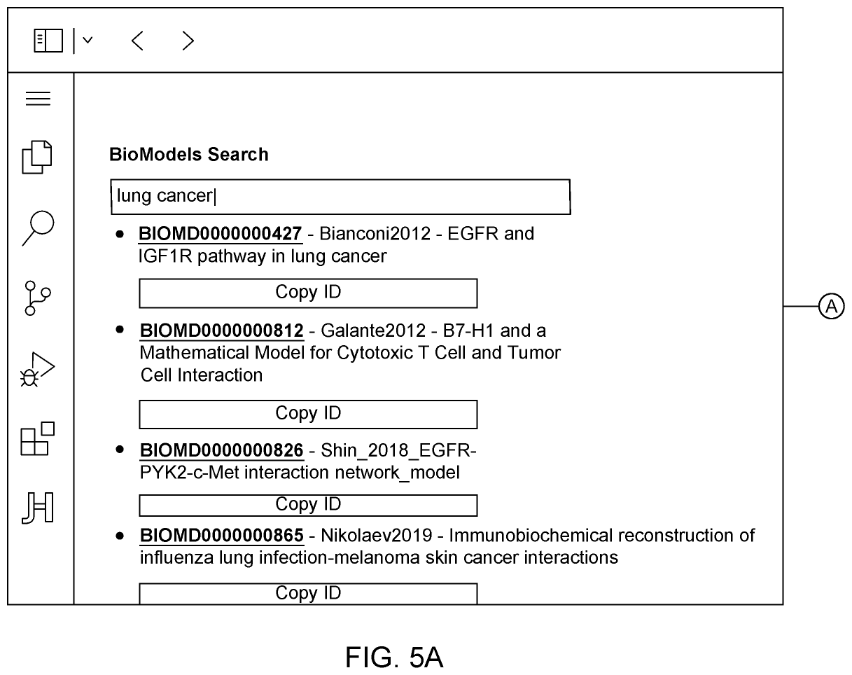

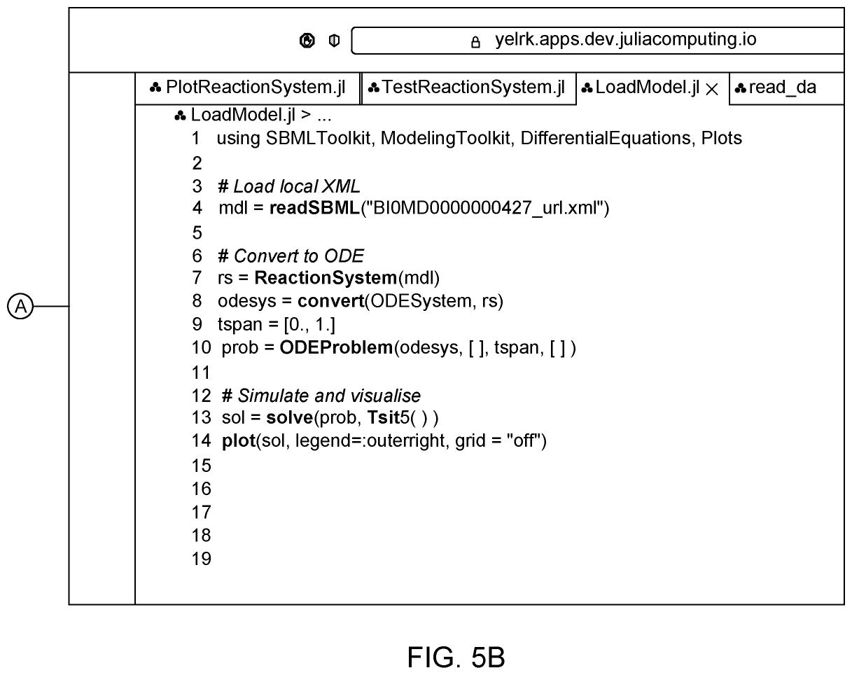

In some cases, these models may be provided by common interexchange formats, such as CellML or SBML. FIGS. 5A and 5B, for example, depict exemplary source code and software extension for loading or importing models according to an embodiment of the subject matter described herein. Referring to FIGS. 5A and 5B, a model may be retrieved from SMBL BioModels or CellML Physiome remote locations or, alternatively, from a local storage location. By importing or loading models as shown, the chance of errors by manual translation may be reduced and less time required.

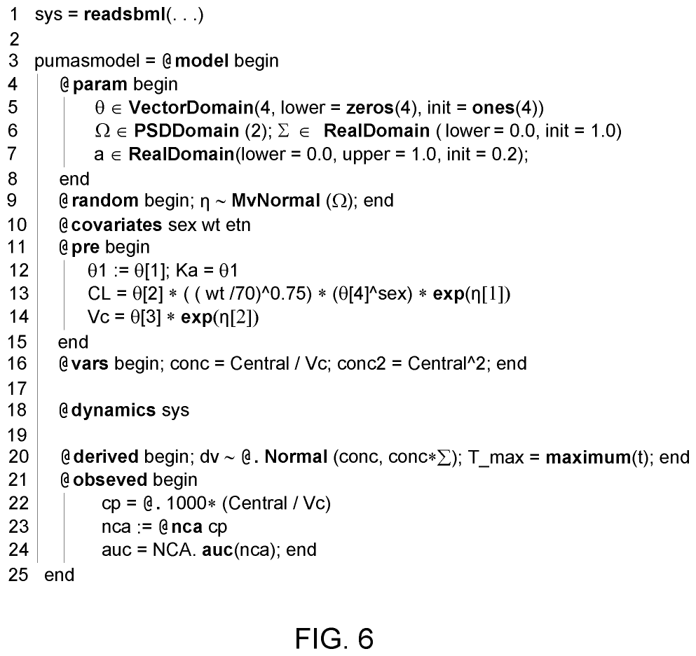

These model specifications (such as those shown in FIGS. 3-4 and 5A-5B, for example) may match those required by nonlinear mixed effects models (NLME), allowing users to easily bridge between such software for what is known as iPSP. FIG. 6, for example, depicts exemplary computer-executable source code for Pumas-QSP iPSP and integration with Pumas according to an embodiment of the subject matter described herein.

These trials additionally encode how some runs of the model may be different from others. For example, in Trial 2, a much higher starting dose may be used, because the dataset with which Trial 2 is paired comes from a study with a higher dose than Trial 1, which is still the same drug. There are many trial types to pair with different data types.

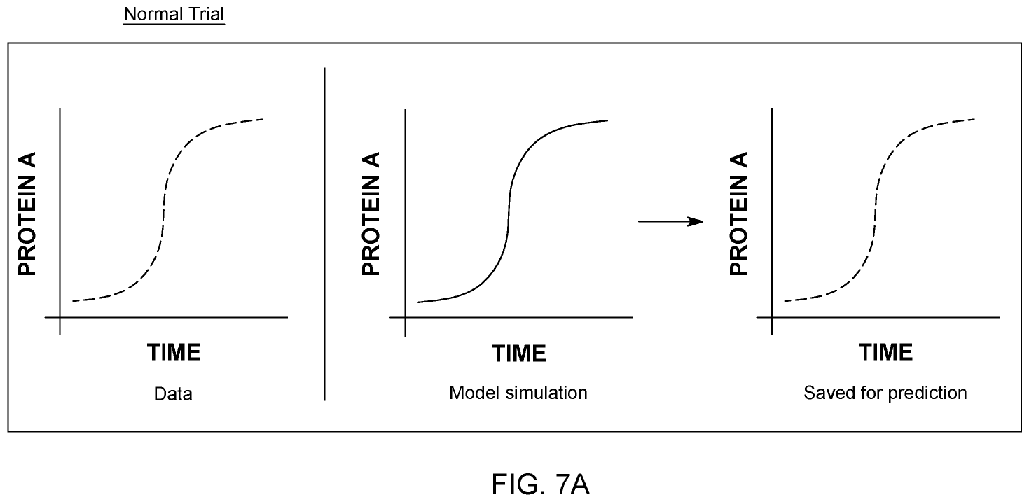

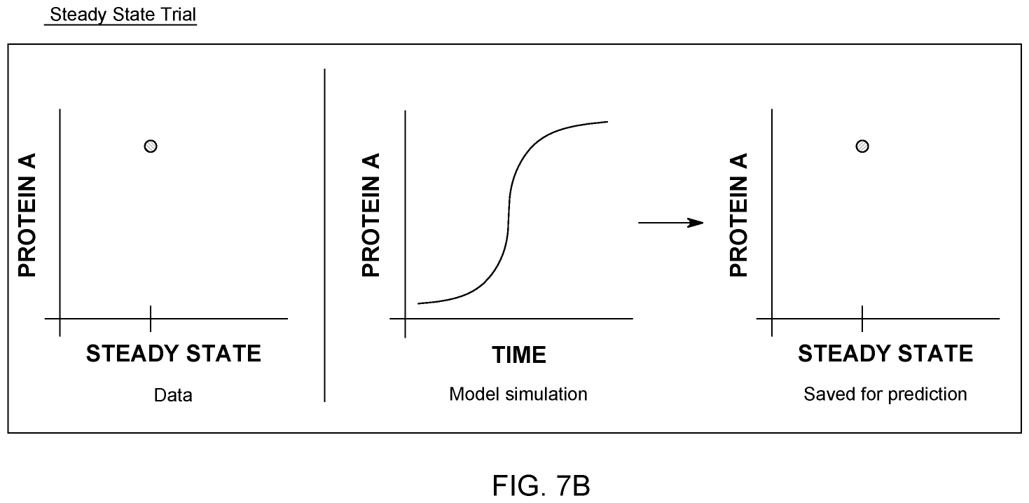

FIGS. 7A and 7B depict, for example, charts illustrating data, model simulation, and predictions for different trial types including a normal trial and a steady state trial according to an embodiment of the subject matter described herein. FIG. 7A depicts data, model simulation, and predictions for a normal trial. FIG. 7B depicts data, model simulation, and predictions for a steady state trial.

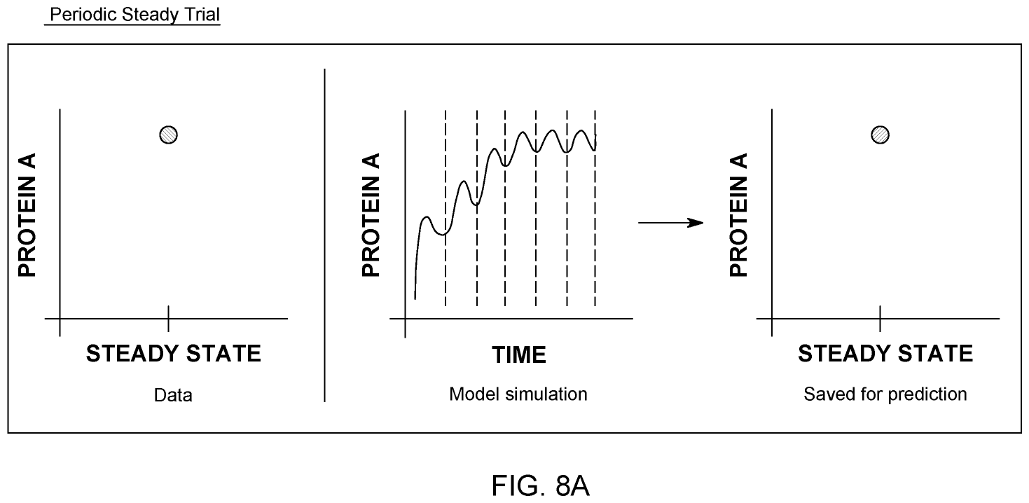

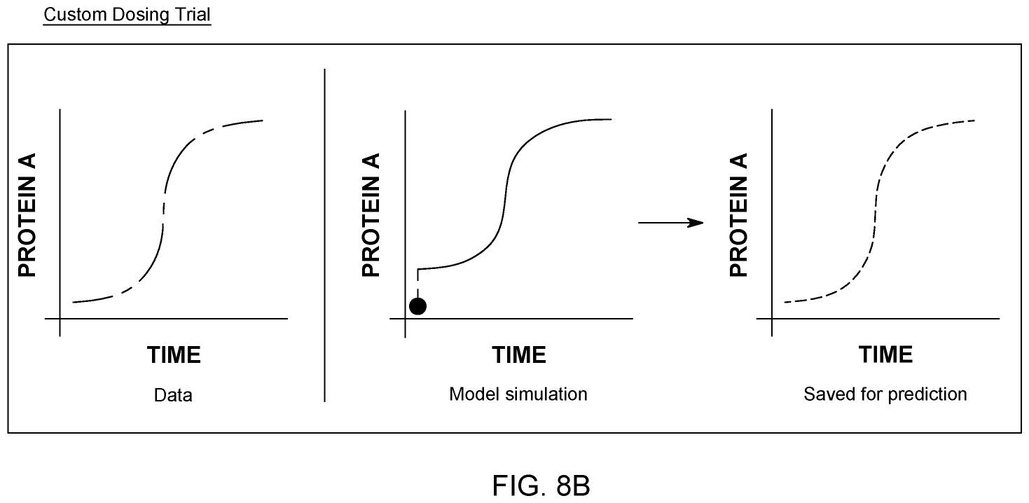

FIGS. 8A and 8B depict, for example, charts illustrating data, model simulation, and predictions for different trial types including a periodic steady state trial and a custom dosing trial according to an embodiment of the subject matter described herein. FIG. 8A depicts data, model, simulation, and predictions for a periodic steady state trial. FIG. 8B depicts data, model, simulation, and predictions for a custom dosing trial.

A standard or normal trial (as represented in FIG. 7A) matches a timeseries of a model solution against timeseries data. While steady state trials (as represented in FIG. 7B) compare against a single endpoint value at steady state, periodic steady state trials (as represented in FIG. 8A) perform a repeated perturbation at regular intervals until convergence, and take the point at the end of the interval. Custom dosing trials (as represented in FIG. 8B) allow for IV Bolus (instantaneous) changes/dosing and infusions, which add a constant rate to the differential equation for fixed duration. Custom reduction functions allow for measuring custom outputs from the trials as well, for example a standard trial produces a time series, but a reduction may reduce this output to simply the mean or maximum.

These trials are paired into a trial collection, which can have dependencies. For example, the starting point for Trial 2 may be the endpoint of Trial 1, where Trial 1 was a periodic steady state trial. This would model the case where a patient population is put on a drug for an extended time with a standard dosing schedule, and then Trial 2 would be a trial on patients who have already had steady usage over a given time period.

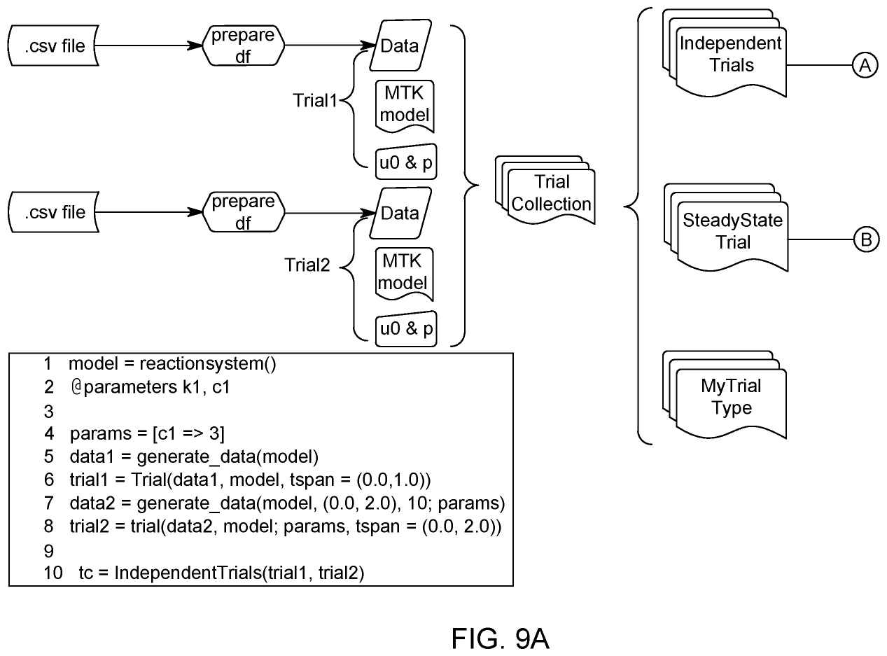

These trials and trial collections are paired with a description of parameter spaces, which, in various embodiments, is a hypercube of maximums and minimums, of the possible values for the parameters for the model. This designs either a QSPCost for generating virtual populations, or a QSPSensitivity for generating global sensitivity analysis results. This is done by calling vpop or gsa with the respective QSP object and an algorithm, for example, vpop(cost::QSPCost,StochGlobalOpt( )) in Julia code. Then algorithms for calculating virtual populations and global sensitivities are run against these collections of trials. The general process is depicted in FIGS. 9A and 9B.

FIGS. 9A and 9B depict a flow chart illustrating exemplary steps in a process for determining trial collections and types according to an embodiment of the subject matter described herein. Referring to FIG. 9A, one or more trials may be grouped together as a trial collection. The trial collection may include, for example, one or more independent trials, steady state trials, or other trial types. Referring to FIG. 9B, the independent trials may include multiple individual trials, where each trial is associated with a timespan, parameters, and u0 (as shown in FIG. 9A and 9B). Each of these values may be combined with a trial cost as input to a setup_trial function.

In the general case, a high-level set of parameters is used to specify values to the trials. Any given values in a trial override these defaults, so, for example, the starting dose may be a quantity being optimized, but it may be one of the values fixed in Trial 2, and therefore the Trial 2 override value is used. This produces a set of predicted values for each of the trials given a global set of parameters.

In virtual populations, the goal is to calibrate the set of global parameters with respect to the datasets included in each of the various trials. For example, if there are 20 trials where 10 have a starting dose parameter override and 10 do not, then the virtual population seeks to calibrate the starting dose parameter to the 10 trials where it is not pre-specified. The parameters can be optimized all simultaneously or sequentially. The parameters are optimized with respect to a cost function that is specified per trial. For each trial, the cost is calculated by a function between the prediction and the data, defaulting to using the Euclidean distance but can be overridden by the user. The total cost is a reduction of the per-trial costs, normally a summation. Each calibrated set of parameters (i.e., a set of global parameters for which the total cost is sufficiently small) is called a virtual patient, and a virtual population is a collection of virtual patients.

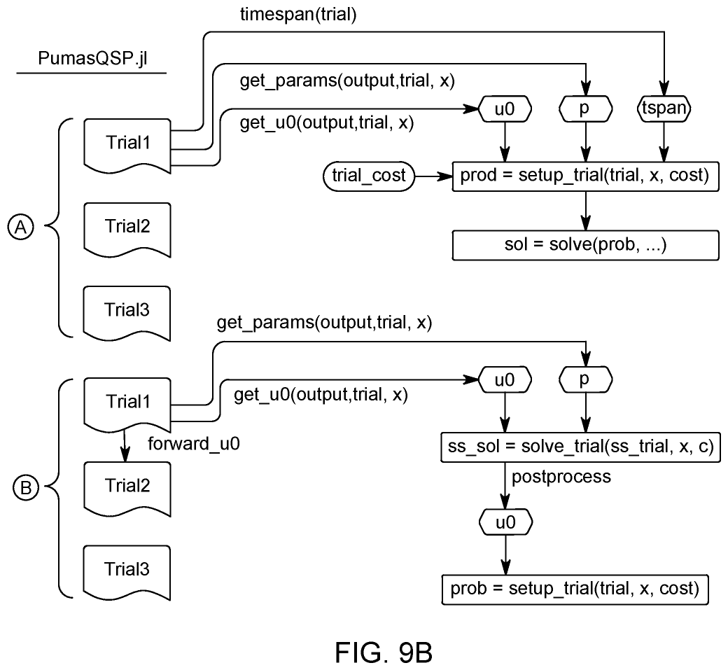

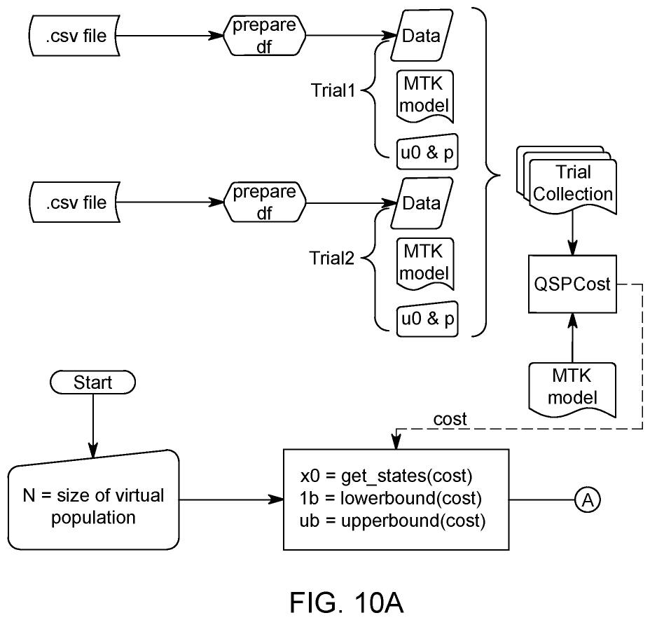

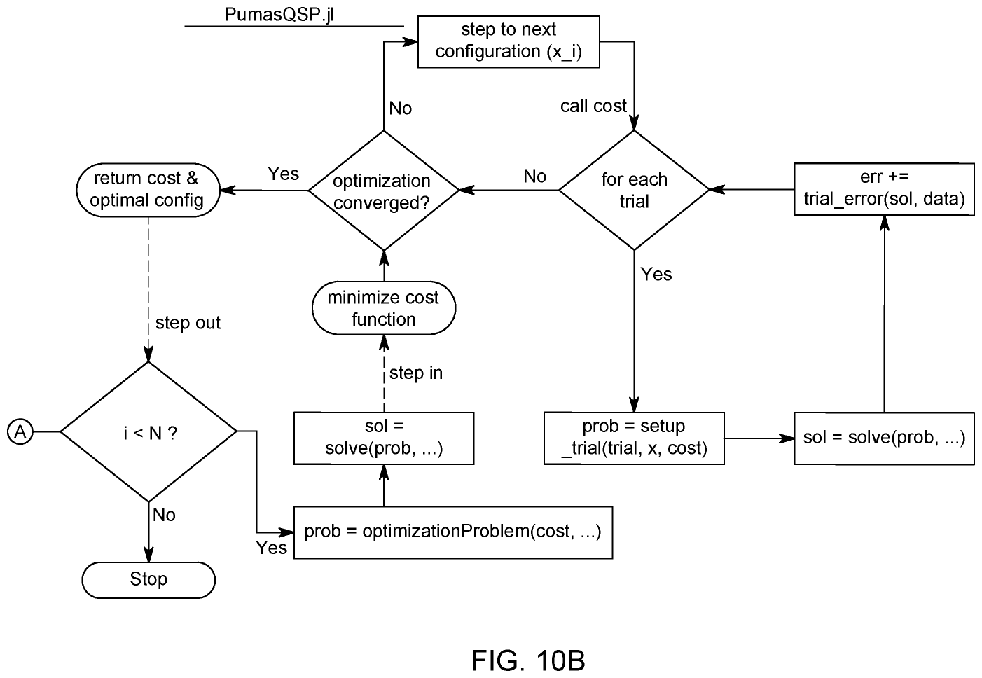

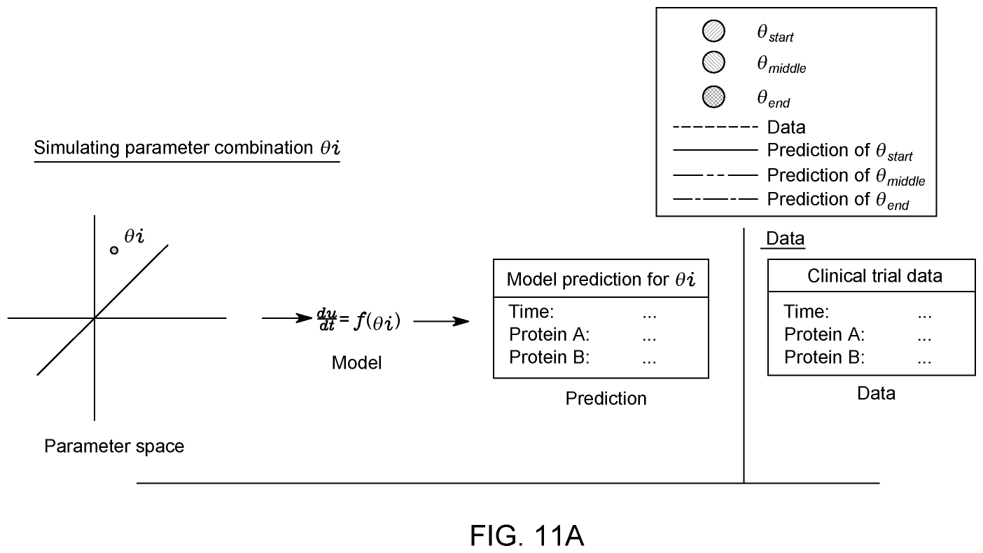

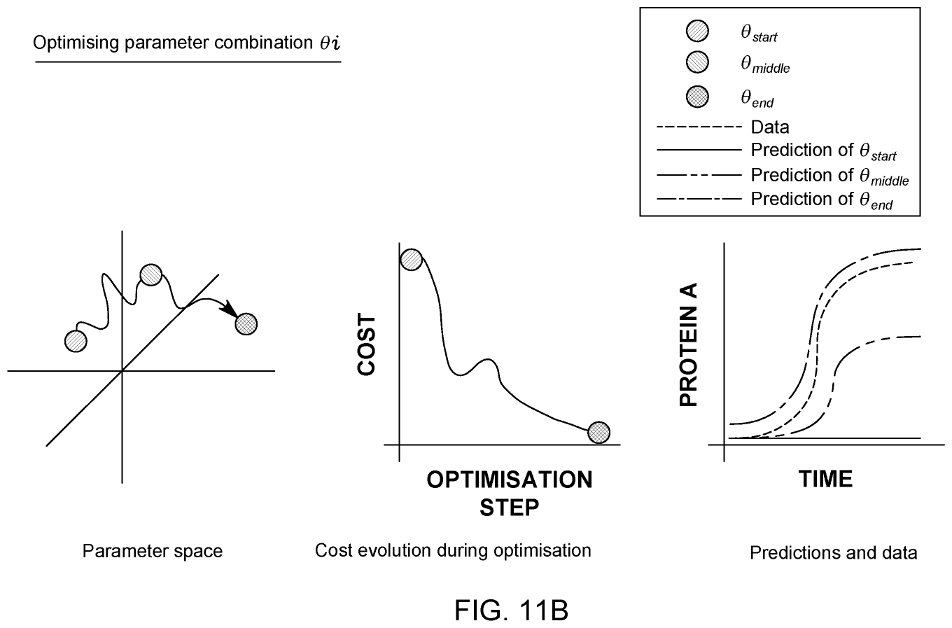

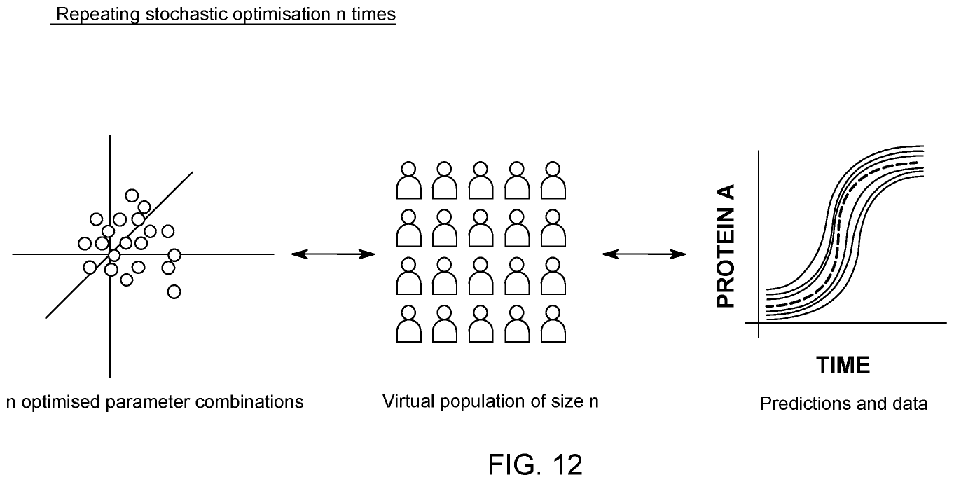

FIGS. 10A and 10B depict a flow chart illustrating an exemplary process for generating virtual populations. This is referred to as StochGlobalOpt, which uses a stochastic global optimization algorithm (such as differential evolution, genetic algorithm, particle swarm, multi-start methods such as TikTak, etc., for which the GalacticOptim.jl package is used) to find a given parameter which receives a sufficiently optimal cost. Each run of these types of global optimization algorithms can find different optima in the space of parameters, providing a set of parameters which all, when put into the set of models, sufficiently fit the data for each of the trials simultaneously. This optimization process at a per-trial level is depicted in FIGS. 11A and 11B, to receive the virtual population (FIG. 12).

Referring to FIGS. 10A and 10B, an upperbound cost (ub) and a lowerbound cost (lb) are determined by QSPCost function for the trial collection over the virtual population. Here, the virtual population is size N and the process is iterated over N. A determination is made whether optimization is converged. If optimization is converged, then the process returns the cost and the optimal configuration. If optimization is not converged, the process continues costing each trial and minimizing the cost function.

FIGS. 11A and 11B depicts a schematical overview of the optimization process at a per-trial level. Referring to FIG. 11A, in section 1, a parameter space includes simulating parameter combination θi. The parameter space is modeled, and a model prediction is generated for θi. In the example shown in FIG. 11A, this includes a time, protein A, and protein B. In section 2, clinical trial data is received or inputted. This data also includes time, protein A, and protein B, as shown in FIG. 11A. In section 3, as shown in FIG. 11B, the parameter combination θi is optimized, which includes examining a cost evolution during optimization and evaluating multiple predictions for each protein over time.

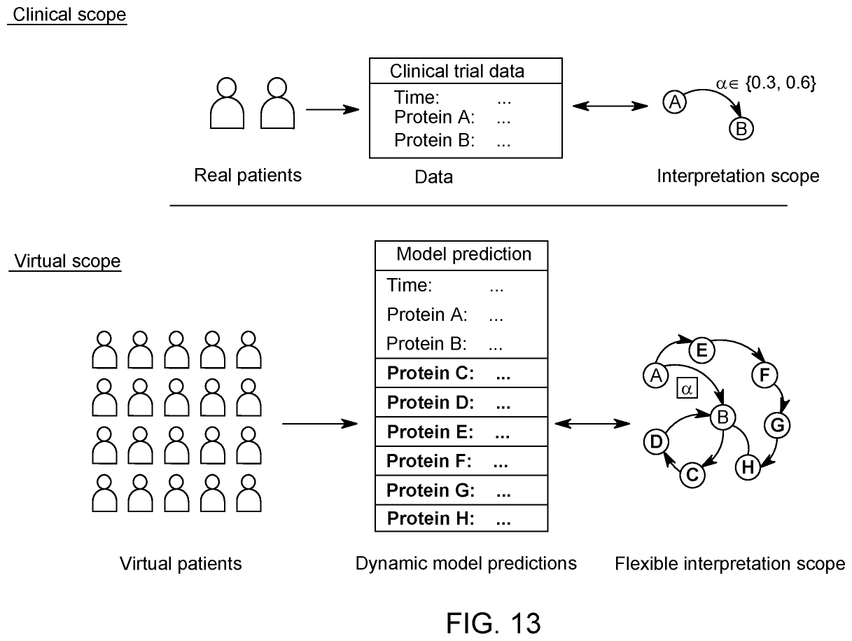

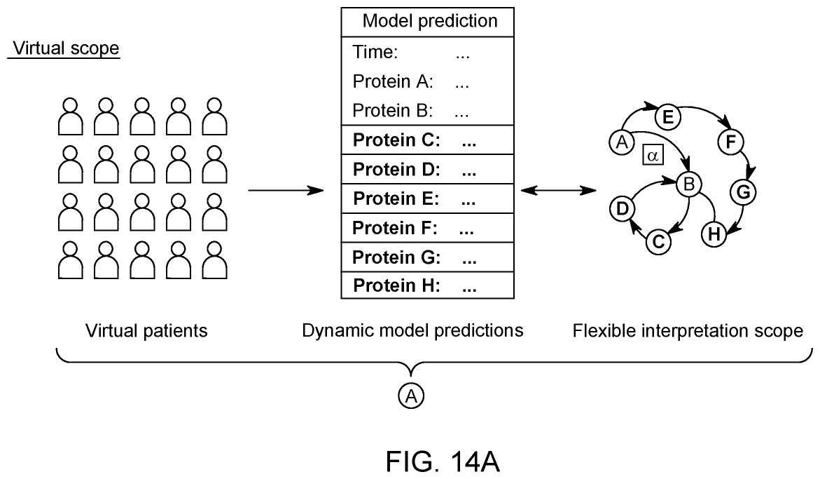

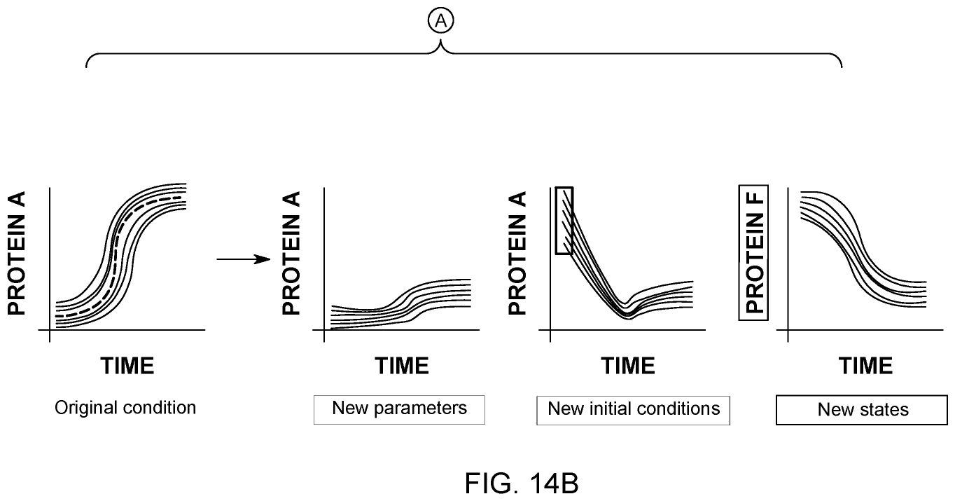

FIG. 12 depicts a schematical overview of receiving the virtual population generated as shown in FIGS. 11A and 11B. Referring to FIG. 12, once VPs are generated given some clinical data, the VPs are used to expand the analysis scope to dimensions where, for example, experimental data collection is difficult. This objective is schematically shown, for example, in FIGS. 13 and 14A-14B. Here, groups include:

(1) The VP to model the system for new values of model parameters. Here, model parameters do not refer to parameters that specify a patient, but other, independent parameters of the model. As an example, we have the reaction rate (alpha).

(2) The VP can be used to model the system for new initial conditions for the observed states of the system. Here, this relates to Protein A and B.

(3) The VP can be used to model states of the system that have not been observed. These are Protein C to H in this example.

Virtual population objects are stored as a collection of optimized parameters, the VirtualPopulation object. Various sub-sampling algorithms, such as MAEPL (from Schmidt et al., “Alternate virtual populations elucidate the type I interferon signature predictive of the response to rituximab in rheumatoid arthritis”, available at https://pubmed.ncbi.nlm.nih.gov/23841912/, the entire contents of which is incorporated by reference herein), or removal of outliers, or rejection sampling techniques, can be used to reduce the virtual population from the raw form received by the virtual population generating process to a form that further matches the high-level statistics of a given dataset or prior knowledge. In some cases, the literature would call the set received by the optimization process the potential patient population, and then the subsampled form as the virtual population, though programmatically, a naming distinction is not necessary (but can be implemented).

From a virtual population (or patient population) virtual trials may be run by specifying perturbations to the parameters of the trials, where any unspecified value then uses the parameter values of a given virtual patient. In code form, this is of a form virtual_trial(vpop::VirtualPopulation, perturbations) where perturbations is a set of maps between symbols (for initial conditions, parameters, etc.) and values.

The subject matter described herein enables users to consider additional model exploration based on global sensitivity analysis (GSA). This approach is helpful to further investigate how the model output reacts to variance in the model input. For example, how the variance in single input variables affects the output variables can be analyzed quantitatively.

FIG. 13 depicts a virtual trial, perturbing a value alpha and allowing the researchers to see the effect on unmeasured quantities. Referring to FIG. 13, a comparison between a clinical scope of relationships between real patients, data, and interpretation scope, on the one hand, and a virtual scope of relationships between virtual patients, dynamic model predictions, and flexible interpretation scope, on the other hand, is shown.

FIGS. 14A and 14B showcase how the virtual trial returns a set of predictions for the perturbed trial for the set of patients. These virtual trials may include visualization tooling for depicting the predictions, as shown in FIGS. 14A and 14B. Referring to FIGS. 14A and 14B, the influence of multiple input variables can be seen to affect the output variables in the example from FIG. 13.

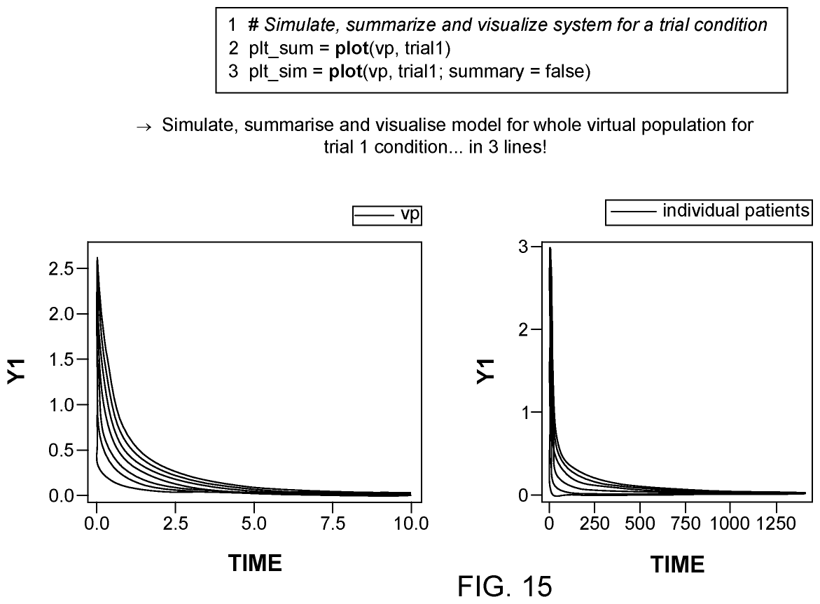

FIG. 15 depicts exemplary source code and a virtual trial output. Referring to FIG. 15, exemplary reporting source code and graphs for simulating, summarizing, and visualizing a model for a virtual population for one trial condition according to an embodiment of the subject matter described herein. This is an example of simulating, summarizing, and visualizing a model for a whole virtual population for a trial condition in just three lines of code.

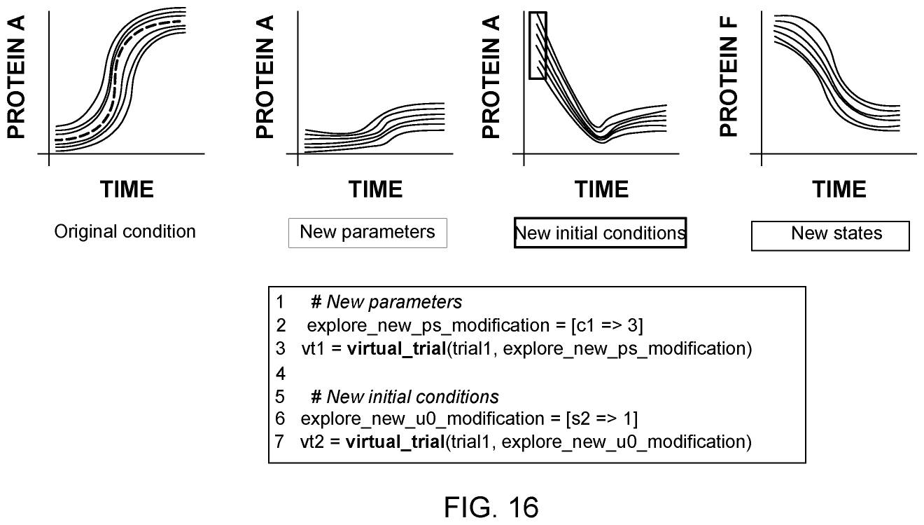

FIG. 16 depicts additional exemplary source code and a virtual trial output. Referring to FIG. 16, exemplary source code and graphs for exploring a system in new settings via virtual trials according to an embodiment of the subject matter described herein.

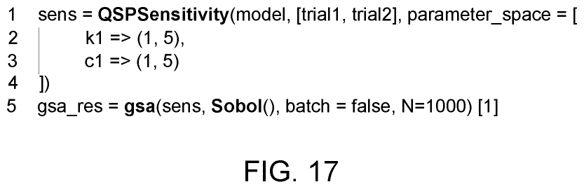

FIG. 17 depicts a similar workflow for global sensitivity analysis (GSA), where a GSA method is computed on the outputs of the trials collected into the QSPSensitivity object. FIG. 17 depicts exemplary computer-executable source code for performing global sensitivity analysis (GSA) according to an embodiment of the subject matter described herein.

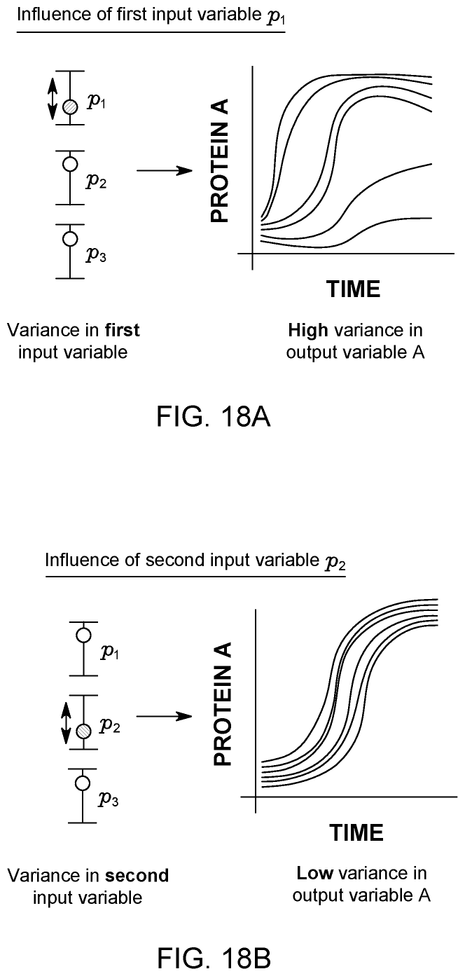

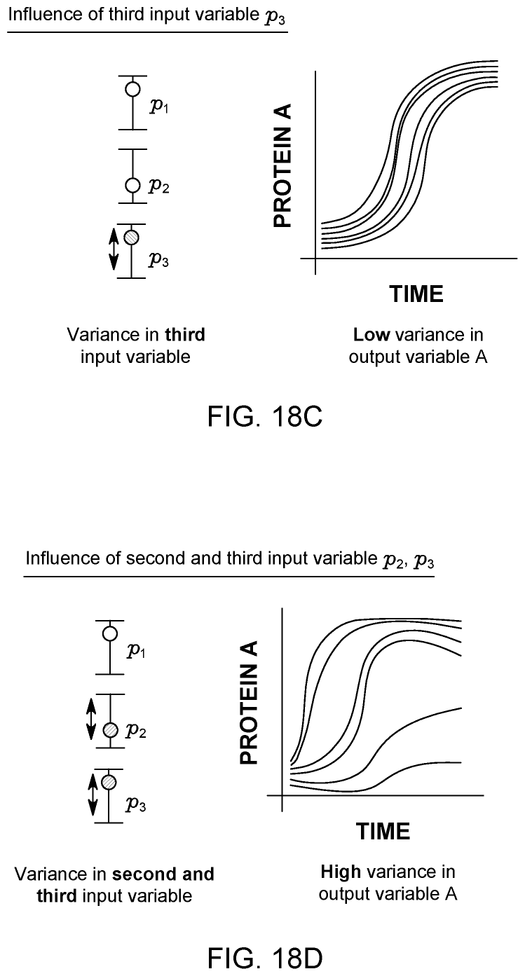

FIGS. 18A-18D describe what global sensitivity analysis (GSA) methods measure. For example, FIG. 18A depicts the influence of variance of the first input variable, where the variance leads to a high variance in the output variable. FIG. 18B depicts the influence of variance of the second input variable, where the variance leads to a low variance in the output variable. FIG. 18C depicts the influence of variance of the third input variable, where the variance leads to a low variance in the output variable. FIG. 18D depicts the influence of variance of the second and third input variables, where the variance leads to a high variance in the output variable.

In an embodiment, the GSA and virtual population calls may be accelerated by using surrogates. These surrogates are models that can be trained to match the computational model to replace it before being used in the virtual population and GSA calls. For example, a CTESN or the like may be used to speed up the processing.

As described herein, the interface design allows for the virtual population and the GSA training processes to be highly parallel operations. In one embodiments, these analyses may be run, for example, using a cloud architecture for parallelizing the analyses across multiple CPUs and GPUs.

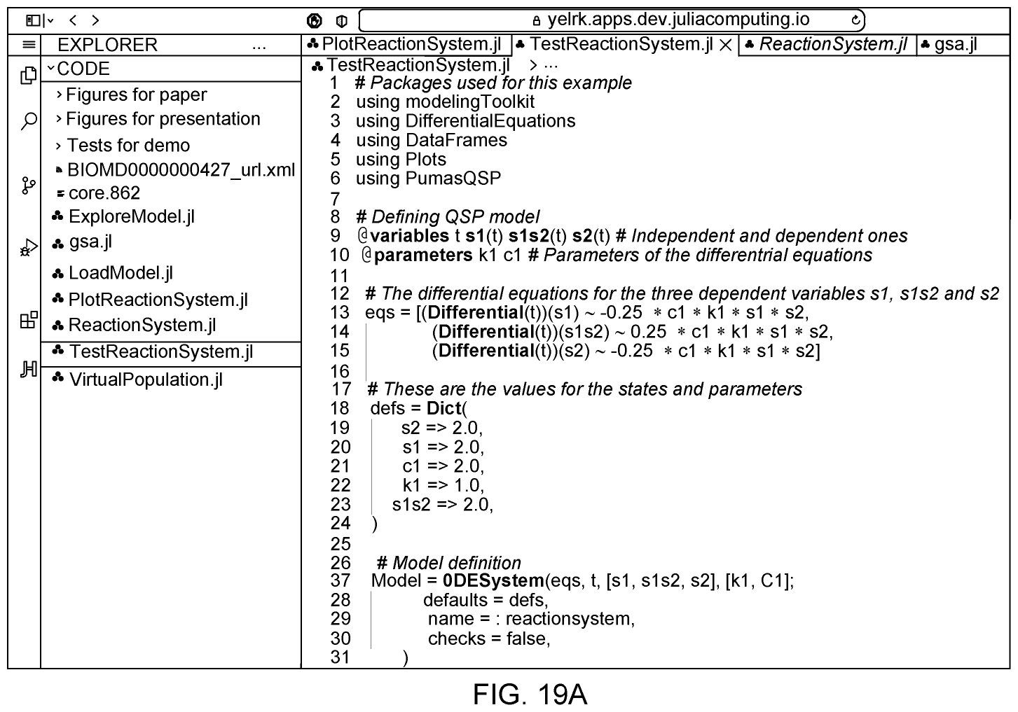

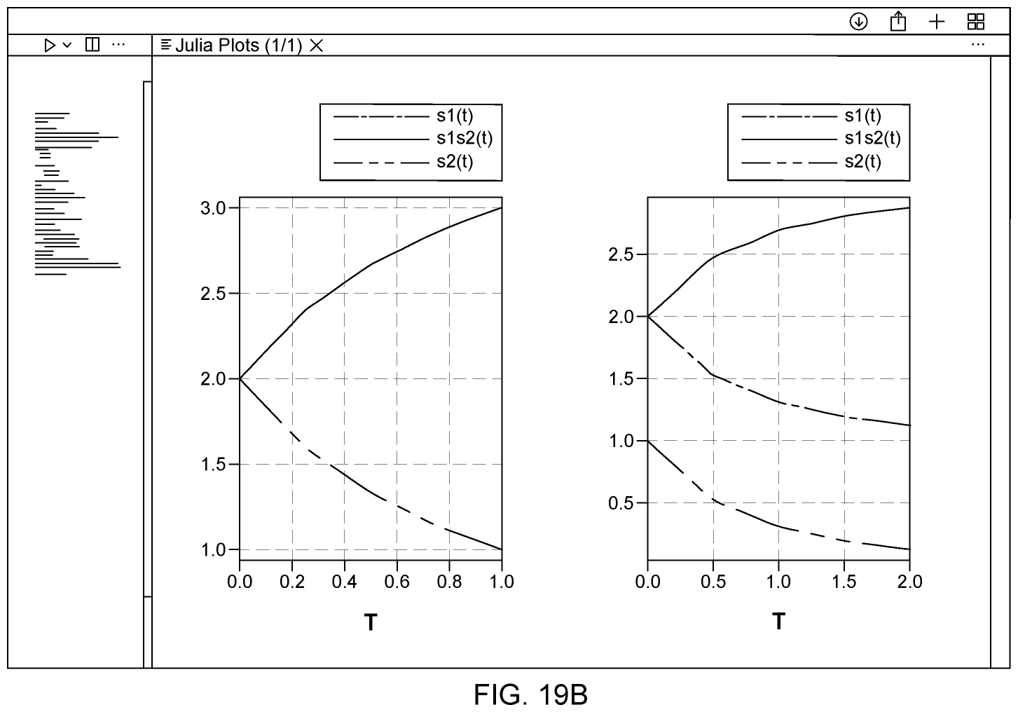

FIGS. 19A and 19B depict an exemplary user interface of JuliaHub implementing Pumas-QSP according to an embodiment of the subject matter described herein. Referring to FIGS. 19A and 19B, the cloud interface of this QSP module running on the JuliaHub cloud platform, where users have access to an essentially unrestricted amount of computing resources. Such a system is compatible with cloud systems like AWS, Google Cloud Compute, and Azure. Users may specify the amount of compute directly by opening a given amount of compute (via addprocs(N) for N virtual CPU processors, for example), or the algorithms can infer a, optimal amount of compute using heuristics and preliminary measurements of trial compute times.

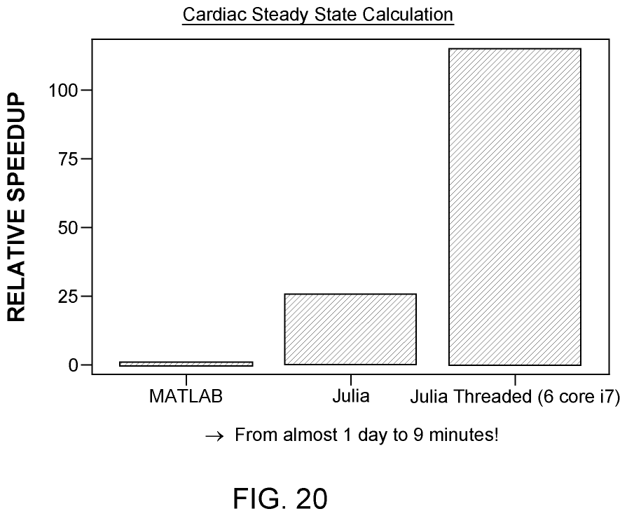

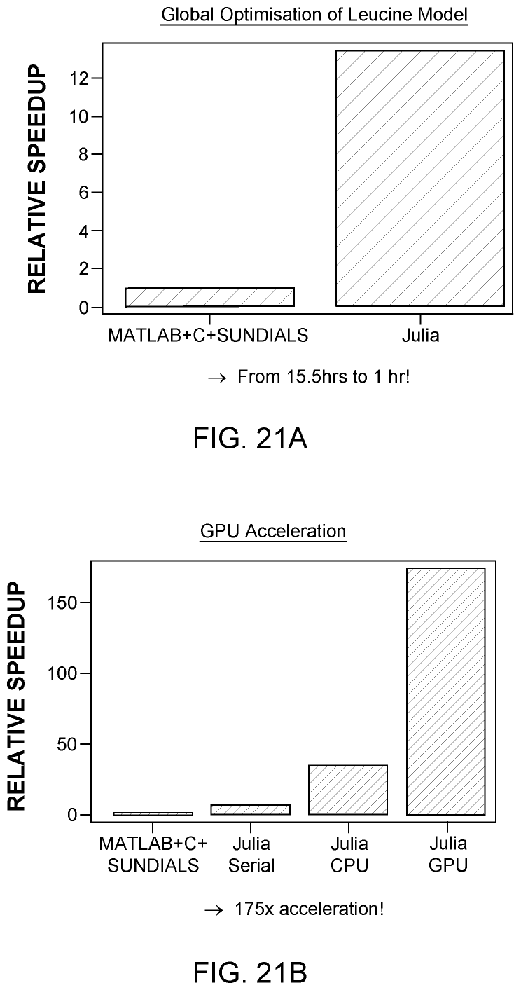

FIGS. 20 and 21A-21B depict the acceleration of virtual population generation, which may be accomplished through parallelization. In particular, FIG. 20 depicts a bar chart illustrating relative acceleration of a large simulation for a cardiac steady state calculation when using Pumas-QSP as described herein versus Matlab. As can be seen from FIG. 20, using the methods and systems disclosed herein results in a reduction in time required to perform the cardiac steady state calculation from one day down to nine minutes, as compared with using Matlab.

FIGS. 21A and 21B depict bar charts illustrating relative acceleration of a large simulation for a global optimization of a Leucine model and GPU acceleration when using Pumas-QSP as described herein versus Matlab+C+Sundials. As can be seen from FIG. 21A, using the methods and systems disclosed herein results in a reduction in time required to perform global optimization of a Leucine model from 15.5 hours down to 1 hour, as compared with using Matlab+C+Sundials. Similarly, as can be seen from FIG. 21B, using the methods and systems disclosed herein with GPU acceleration results in a 175× speedup relative to Matlab+C+Sundials.

As mentioned above, in QSP, the systems of interest grow quickly in scope, and collecting enough clinical trial data is often infeasible or even impossible. A virtual population (VP) addresses this imbalance of model complexity and available data. Each model parameter combination that describes given data sufficiently well is considered a virtual patient. A collection of virtual patients is considered a virtual population. Virtual populations emulate the diversity in patients and can be used to explore variability in effects in clinical trials.

The creation of VPs may be connected to unidentifiable functions. A function f:X→Y is identifiable, if, and only if, for all x in X, f(x)=y implies that there exists no x′ where f(x′)=y. If the output generation of a QSP model is thought of as a non-identifiable f, and y is the given observed trial data, a VP consists of parameter combinations x′ that can produce y (with some error tolerance). The PumasQSP.jl package provides a methodological framework to create VPs in Julia using stochastic optimization.

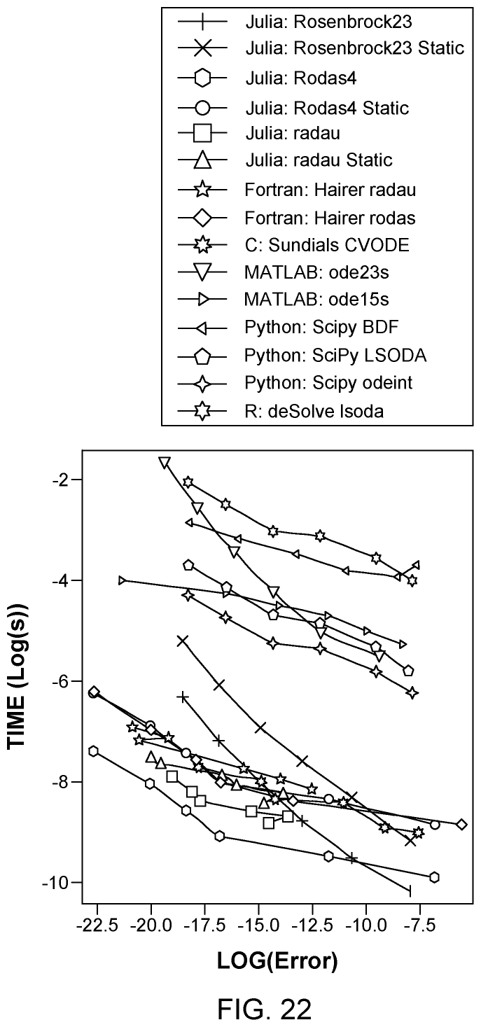

FIG. 22 depicts a line graph of the relationship between time and error for fast model simulations performed by DifferentialEquations.jl for exemplary Hires equations of nine stiff chemical reactions. As can be seen from FIG. 22, the error for the Julia simulations of the chemical reactions is lower than for existing methods.

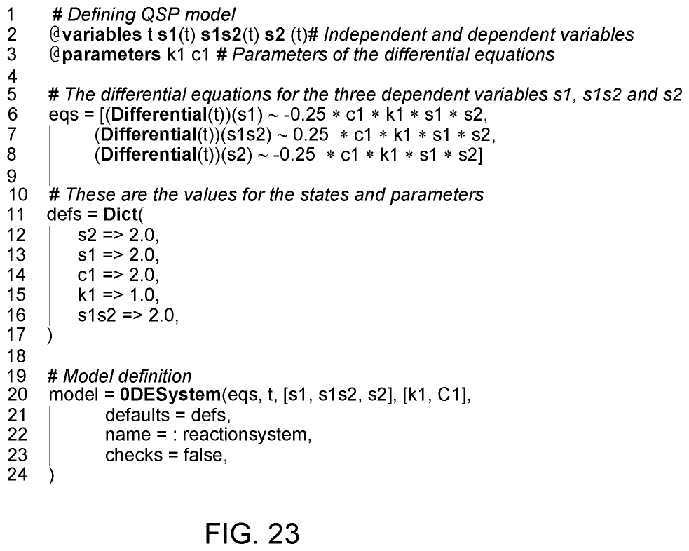

FIG. 23 depicts exemplary computer-executable source code for defining a QSP model with ModelingToolkit.jl.

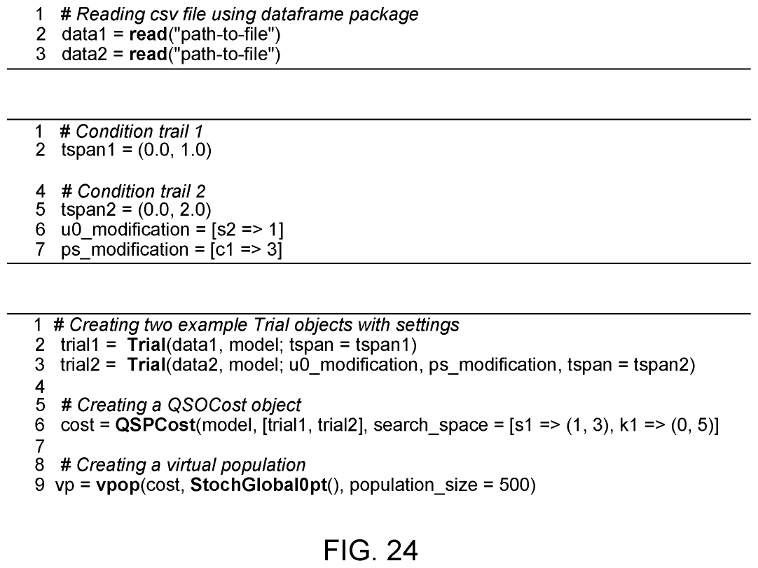

FIG. 24 depicts exemplary computer-executable source code for reading and analyzing trial data according to an embodiment of the subject matter described herein.

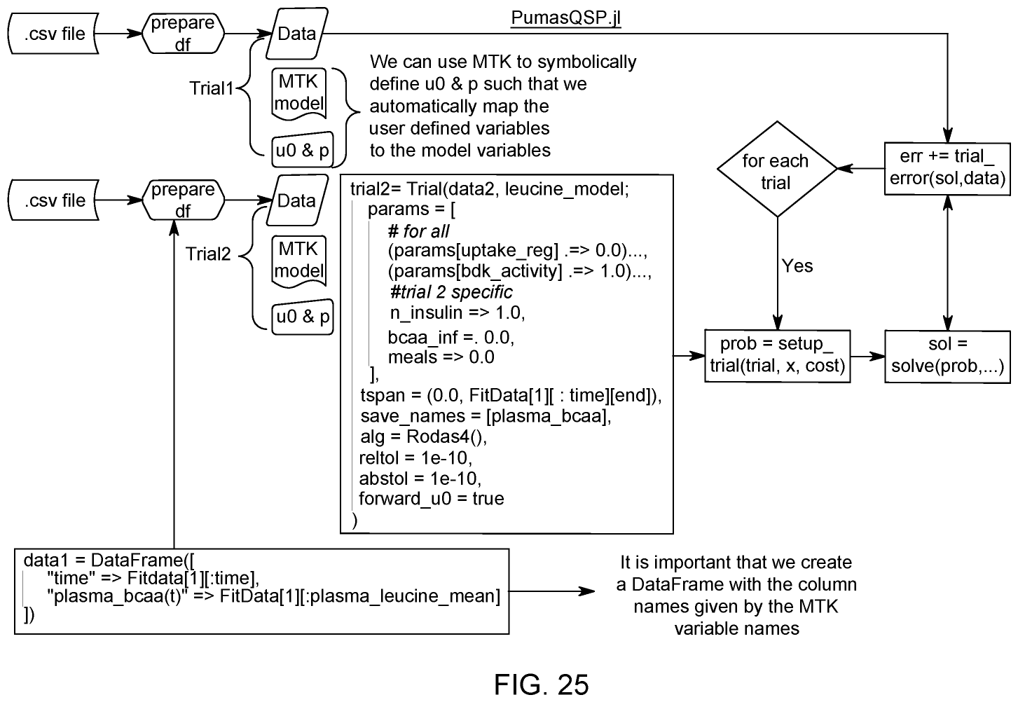

FIG. 25 depicts a flow chart illustrating exemplary steps in a process for receiving user input according to an embodiment of the subject matter described herein. Referring to FIG. 25, data may be received as a csv file and a data frame is prepared. The data, an MTK model, and mappings u0 and p may collectively determine a first trial (Triall). The MTK can be used to symbolically define u0 and p such that user-defined variables can be automatically mapped to the model variables.

A second trial (Trial2) is illustrated with associated example source code. It is appreciated that a DataFrame is created with column names given by the MTK variable names. The data from the trials may be used to determine error for each trial and a probability.

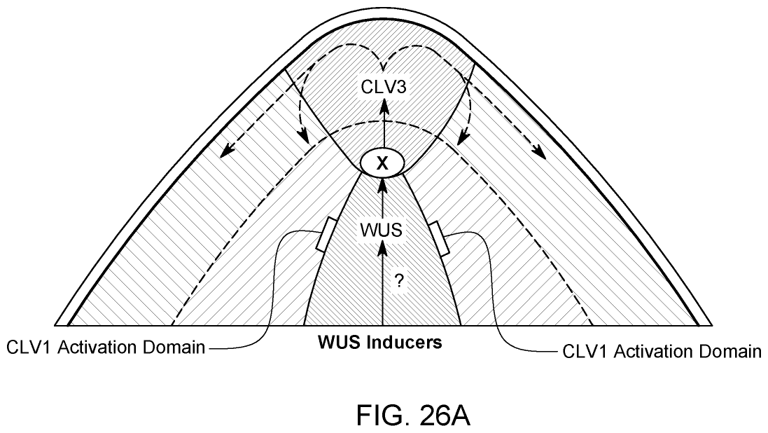

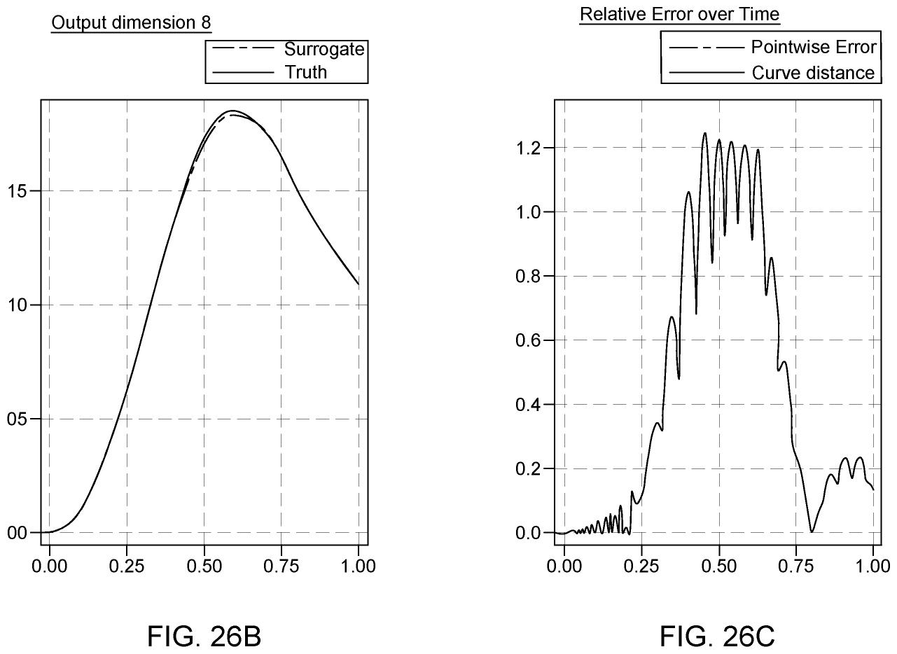

FIGS. 26A-26C depict charts illustrating AI acceleration modeling of QSP models, including an exemplary Arabidopsis model, when using Pumas-QSP according to an embodiment of the subject matter described herein. Additionally, continuous-time echo state network (CTESN) may be used as surrogates to speed up modeling.

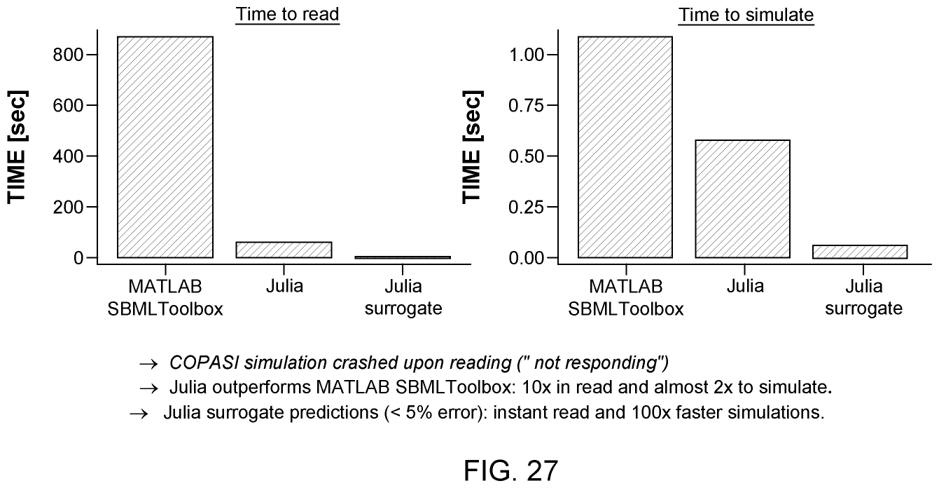

FIG. 27 depicts bar charts illustrating the relative speedup of an exemplary Arabidopsis model when using Pumas-QSP versus Matlab SBML Toolbox.

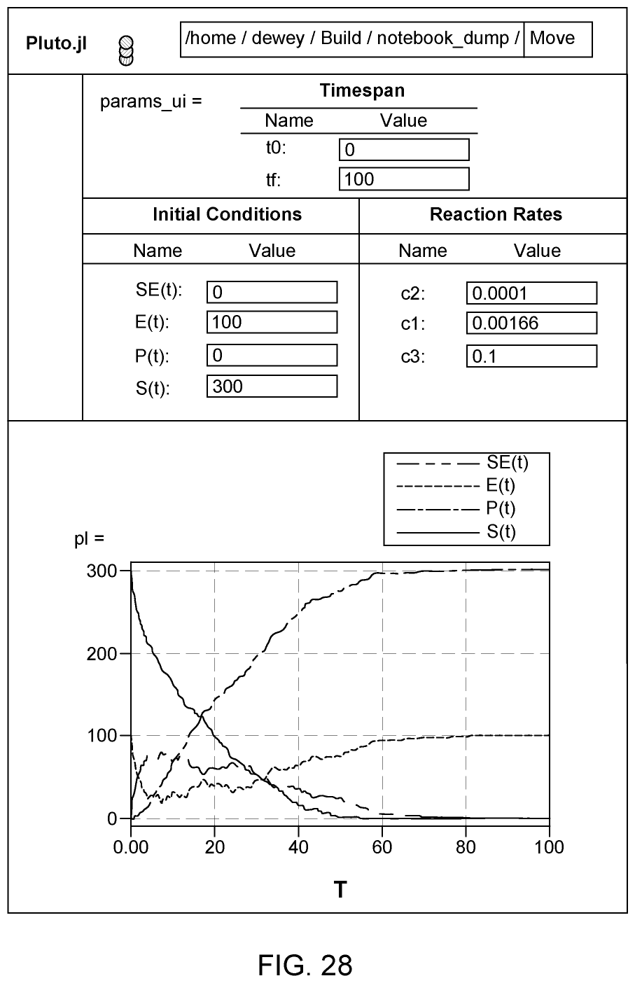

FIG. 28 depicts an exemplary dynamic and automatic report generated according to an embodiment of the subject matter described herein. Referring to FIG. 28, it is appreciated that the dynamic and automatic reporting in the Julia environment may include a user-specific visualization, such as shown which includes an indication of the applicable timespan, initial condition, and reaction rates that produce the color-coded line graph.

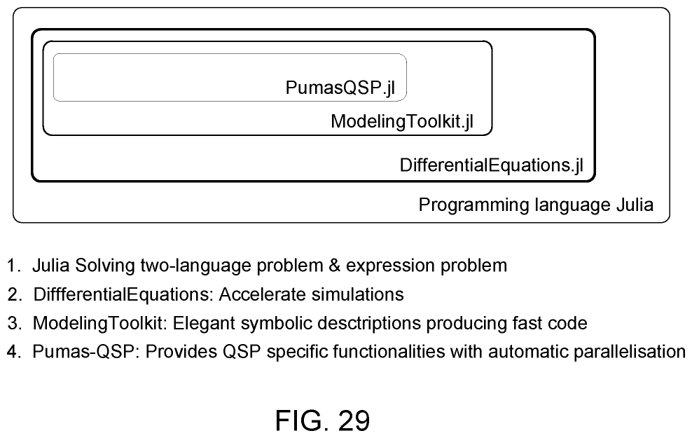

FIG. 29 depicts a functional relationship between various software modules of the subject matter described herein.

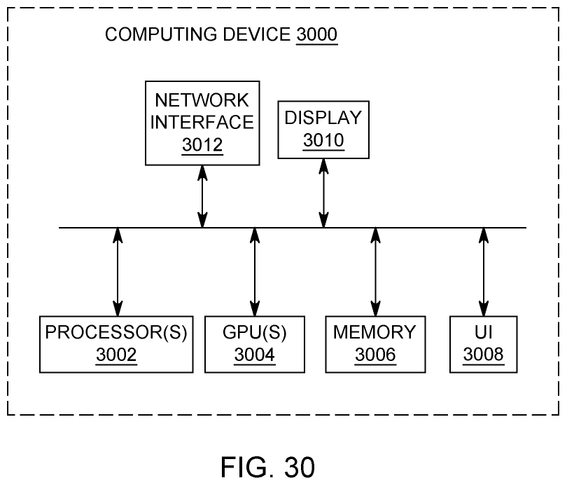

FIG. 30 depicts a block diagram illustrating one embodiment of a computing device that implements the methods and systems described herein. Referring to FIG. 30, the computing device 3000 may include at least one processor 3002, at least one graphical processing unit (“GPU”) 3004, a memory 3006, a user interface (“UI”) 3008, a display 3010, and a network interface 3012. The memory 3006 may be partially integrated with the processor(s) 3002 and/or the GPU(s) 3004. The UI 3008 may include a keyboard and a mouse. The display 3010 and the UI 3008 may provide any of the GUIs in the embodiments of this disclosure.

### Using PumasQSP.jl: Example Use Case—Generating Virtual Populations

An example use case demonstrating PumasQSP.jl for a simple QSP modelling case is provided below. It is appreciated that a QSP model, clinical trial data, and cost specifications may be necessary to create VPs with PumasQSP.jl.

# Packages used for this example
using PumasQSP
using ModelingToolkit
using OrdinaryDiffEq
using DataFrames
using Plots

This example use case considers a QSP model with three variables and two parameters, the dynamics of which are described by three equations and default values are set for the variables and parameters. ModelingTookit.jl package is used to specify the model in the Julia programming language.

```
# Defining QSP model
@variables t s1(t) s1s2(t) s2(t) # Independent and dependent variables
@parameters k1 c1 # Parameters of the differential equations
# The differential equations for the three dependent variables s1,
s1s2 and s2
eqs = [(Differential(t))(s1) ~ −0.25 * c1 * k1 * s1 * s2,
    (Differential(t))(s1s2) ~ 0.25 * c1 * k1 * s1 * s2,
    (Differential(t))(s2) ~ −0.25 * c1 * k1 * s1 * s2]
# These are the values for the states and parameters
defs = Dict (
    s2 => 2.0,
    s1 => 2.0,
    c1 => 2.0,
    k1 => 1.0,
    s1s2 => 2.0,
)
# Model definition
model = ODESystem(eqs, t, [s1, s1s2, s2], [k1, c1];
    defaults = defs,
    name = :reactionsystem,
    checks = false,
  )
```

The model is described by an ODE system. Simulating the model produces time series data over a given time frame and this process relates to simulating parameter combination (theta_i).

One way to manage clinical trial data is to use the package DataFrames.jl to read data easily and efficiently.

# Reading csv file using dataframe package
data1 = read(<path-to-file>)

This step relates to data, however, for the sake of clarity, we simulate this data in this example.

# Generate two trial data sets
noise_level = 0.5
n = 5
#Data for trial 1
tspan1 = (0.0, 1.0)
probl = ODEProblem(model, [ ], tspan1)
saveat1 = range(prob1.tspan..., length = n)
sol1 = solve(prob1, Tsit5( ); saveat = saveat1)
data1 = DataFrame(sol1)
rd1 = randn (5,3).*noise_level
data1[:, 2:end] .+= rd1
#Data for trial 2
tspan2 = (0.0, 2.0)
u0_modification = [s2 => 1]
ps_modification = [c1 => 3]
prob2 = ODEProblem(model, u0_modification, tspan2, ps_modification)
saveat2 = range (prob2.tspan..., length = n)
sol2 = solve(prob2, Tsit5( ); saveat = saveat2)
data2 = DataFrame(sol2)
rd2 = randn(5,3).*noise_level
data2[:, 2:end] .+= rd2

A visualization of the data may be generated that shows the general dynamic of the model as well as the noise levels.

```
# Visualize the generated Trial data
p1 = scatter(data1[:, 1], data1[:, 2])
  scatter!(data1[:, 1], data1[:, 3])
  scatter!(data1[:, 1], data1[:, 4])
p2 = scatter(data2[:, 1], data2[:, 2])
  scatter!(data2[:, 1], data2[:, 3])
  scatter!(data2[:, 1], data2[:, 4])
s1_p = plot(sol1)
s2_p = plot(sol2)
plot(p1, p2)
plot(s1_p, s2_p)
```

Once the data and QSP model are created, the PumasQSP.jl object Trial is used.

# Creating two example Trial objects with different settings
trial1 = Trial(data1, model; tspan = tspan1)
trial2 = Trial(data2, model; u0_modification, ps_modification,
tspan = tspan2)

Here, a cost function is specified by the PumasQSP.jl object: QSPCost. Next, turning to the model and the trials, the user specifies a search space to constrain the values that the optimizer considers as potential values for the parameter combinations.

# Creating a QSPCost object
cost = QSPCost(model, [trial1, trial2],
search_space = [s1 => (1., 3), k1 => (0, 5)])

Once the QSP model and the trials are created, virtual patients can be generated. This may include finding parameter combinations for the provided model which describe the clinical trial data sufficiently well measured in terms of the specified cost. In one embodiment, this is done based on a stochastic optimization approach. In each optimization step, a new parameter combination is selected, the model is simulated (i.e., the ODE problem is solved), and the ODE solution is compared to the clinical trial data to assess its fit. This process relates to optimizing parameter combination (theta_i).

# Creating virtual population
vp = vpop(cost, StochGlobalOpt( ), population_size = 500)

With the example vpop call shown above, 500 separate optimization runs are started leading to a VP with 500 patients. This relates to repeating stochastic optimization n times. Thus, the user can conveniently analyze and visualize the results by transforming the VP to an EnsembleProblem of the DifferentialEquations.jl suite.

```
# Transform to EnsembleProblem, solve and summarize it
ensembleprob = vpop_ensemble(vp, cost; tspan = (0.0, 2.0))
sim = solve(ensembleprob, Tsit5( ), trajectories = length(vp))
sum = EnsembleSummary(sim)
# Visualization (1/2): Simulation results
P_sim = plot(sum)
  plot!(data1[:, 1], data1[:, 2])
  plot!(data1[:, 1], data1[:, 3])
  plot!(data1[:, 1], data1[:, 4])
  plot!(data2[:, 1], data2[:, 2])
  plot!(data2[:, 1], data2[:, 3])
  plot!(data2[:, 1], data2[:, 4])
# Visualization (2/2): Parameters
pars_s1 = [vp[i][2] for i in 1:length(vp)]
pars_k1 = [vp[i][1] for i in 1:length(vp)]
p_scatter = scatter(pars_s1, pars_k1, label = “optimized values”
  xlab = “s1”, ylab = “k1”, xlim = (1., 3), ylim = (0, 5))
  scatter!([defs[s1]], [defs[k1]], label = “generating value”)
```

### Using PumasQSP.jl: High Level Example Use Case—Global Sensitivity Analysis (GSA)

An example of performing GSA on the Lotka-Volterra model is provided below. It is appreciated that several global sensitivity analysis methods are supported, however, this example focuses on the Sobol method.

# Packages used for this example
using PumasQSP
using GlobalSensitivity
using OrdinaryDiffEq
using ModelingToolkit
using Plots
using Statistics

The Lotka-Volterra model consists of two states and four parameters. The model here is defined as an ODESystem, solved for a fixed parameter combination, and visualized.

```
# Model definition and visualization of one solve
@variables t u[1:2] (t)
@parameters p[1:4]
D = Differential(t)
eqs = [
  (Differential(t))(u[1]) ~ p[1] * u[1] − p[2] * u[1] * u[2]
  (Differential(t))(u[2]) ~ −p[3] * u[2] + p[4] * u[1] * u[2]
]
u0 = [u[1] => 1.0, u[2] => 1.0]
po = [p[1] => 1.5, p[2] => 1.0, p[3] => 3.0, p[4] => 1.0]
tspan = (0.0, 10.0)
sys = ODESystem(eqs, name = :predator_prey, defaults = [u0; p0])
prob = ODEProblem(sys, Pair[ ], tspan, Pair[ ])
saveat = range(tspan..., length = 200)
sol = solve(prob, Tsit5( ); saveat = saveat)
plot(sol)
```

Again, a Trial object is created, and a reduction is implemented where the solution of the ODE system is summarized for each state. Here, this includes the mean for the prey population and the maximum for the predator population which are calculated using the Statistics package. Subsequently, the ranges for the parameters of the model are defined to generate quasi-random samples using the Sobol sequence.

```
# Definition Trial object without data
trial = Trial(nothing, sys;
  tspan,
  saveat = saveat,
  alg = Tsit5( ),
  reduction = sol -> [mean(sol[1, :1]), maximum(sol[2, :])]
)
# Create QSPSensitivity and retrieve sensitivities
sens = QSPSensitivity(sys, [trial], parameter_space = [
  p[1] => (1, 5),
  p[2] => (1, 5),
  p[3] => (1, 5),
  p[4] => (1, 5)
])
gsa_res = gsa (sens, Sobol( ), batch = false, N=1000)[1]
# Visualize GSA results
p_tot_prey = bar([“p[1]”, “p[2]”, “p[3]”, “p[4]”], gsa_res.ST[1, :], title = “Total..”
p_first_prey = bar([“p[1]”, “p[2]”, “p[3]”, “p[4]”], gsa_res.S1[1, :], title = “First..”
p_tot_prey = bar([“p[1]”, “p[2]”, “p[3]”, “p[4]”], gsa_res.ST[2, :], title = “Total..”
p_tot_prey = bar([“p[1]”, “p[2]”, “p[3]”, “p[4]”], gsa_res.S1[2, :], title = “First..”
plot(p_tot_prey, p_first_prey, p_tot_pred, p_first_pred)
```

Aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Computer program code for carrying out operations for aspects of the present disclosure may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present disclosure are described above with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These non-transitory computer program instructions may also be stored in a non-transitory computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the present disclosure. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

The corresponding structures, materials, acts, and equivalents of all means or step plus function elements in the claims below are intended to include any structure, material, or act for performing the function in combination with other claimed elements as specifically claimed. The description of the present disclosure has been presented for purposes of illustration and description, but is not intended to be exhaustive or limited to the present disclosure in the form disclosed. Many modifications and variations will be apparent to those of ordinary skill in the art without departing from the scope and spirit of the present disclosure. The embodiments were chosen and described in order to best explain the principles of the present disclosure and the practical application, and to enable others of ordinary skill in the art to understand the present disclosure for various embodiments with various modifications as are suited to the particular use contemplated.

These and other changes can be made to the disclosure in light of the Detailed Description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

The systems and methods described herein may use a component architecture made up of composable subsystem components. The composable subsystem components may be reusable trained surrogates. As used herein, component-based modeling refers to stitching together a large-scale causal or acausal model using trained surrogates. The composable subsystem components are reusable, such that the subsystem components (i.e., surrogates) have been trained, and a library of the trained surrogates is created, which allows for large-scale models to be built using the trained surrogates, and the trained surrogates provide for automatic acceleration of the model. In this way, complex models are built by stitching together pre-designed, pre-shrunk components consisting of self-contained systems. Thus, using trained surrogates for modeling purposes and accelerated differential-equation solving creates an architecture that can solve and/or simulate complex physical processes that were previously infeasible to solve in a commercially reasonable time.

The systems and methods described herein may provide a library of trained surrogates that can be used and reused by users without specialized experience in scientific computing. In some embodiments, the systems and methods described herein may use GPU computing or distributed parallelism with the surrogates to compute the results even faster.

In some embodiments, neural or universal differential equations are used as surrogates for the components. These models are differential equations that have a learnable nonlinear function, such as a Gaussian process, radial basis function, polynomial basis functions (Chebyshev polynomials, Legendre polynomials, etc.), Fourier expansions, or a neural network. These function representations can be trained to predict accurate timeseries for the dynamics of a given component. In some embodiments, the system of differential equations that is learned is smaller than the original component, making it known as a nonlinear model order reduction.

In some embodiments, surrogates, such as continuous or discrete time echo state networks, may be used to emulate the behavior of a component. Echo state networks are processes which simulate a dynamical reservoir process and learn a projection matrix to recover the dynamics. While this case may not result in a smaller system, this representation can be much more efficient to compute due to having numerical properties such as decreased stiffness.

In some embodiments, the direct computation of the timeseries outputs may be approximated by a surrogate such as a (physics-informed) neural network or radial basis function, providing a mesh-free representation of the time series which can be sampled as necessary within the composed simulation.

The subject matter described herein may include the use of machine learning performed by at least one processor of a computing device and stored as non-transitory computer executable instructions (software or source code) embodied on a non-transitory computer-readable medium (memory). Machine learning (ML) is the use of computer algorithms that can improve automatically through experience and by the use of data. Machine learning algorithms build a model based on sample data, known as training data, to make predictions or decisions without being explicitly programmed to do so. Machine learning algorithms are used where it is unfeasible to develop conventional algorithms to perform the needed tasks.

In certain embodiments, instead of or in addition to performing the functions described herein manually, the system may perform some or all of the functions using machine learning or artificial intelligence. Thus, in certain embodiments, machine learning-enabled software relies on unsupervised and/or supervised learning processes to perform the functions described herein in place of a human user.

Machine learning may include identifying one or more data sources and extracting data from the identified data sources. Instead of or in addition to transforming the data into a rigid, structured format, machine learning-based software may load the data in an unstructured format and automatically determine relationships between the data. Machine learning-based software may identify relationships between data in an unstructured format, assemble the data into a structured format, evaluate the correctness of the identified relationships and assembled data, and/or provide machine learning functions to a user based on the extracted and loaded data, and/or evaluate the predictive performance of the machine learning functions (e.g., “learn” from the data).

In certain embodiments, machine learning-based software assembles data into an organized format using one or more unsupervised learning techniques. Unsupervised learning techniques can identify relationship between data elements in an unstructured format.

In certain embodiments, machine learning-based software can use the organized data derived from the unsupervised learning techniques in supervised learning methods to respond to analysis requests and to provide machine learning results, such as a classification, a confidence metric, an inferred function, a regression function, an answer, a prediction, a recognized pattern, a rule, a recommendation, or other results. Supervised machine learning, as used herein, comprises one or more modules, computer executable program code, logic hardware, and/or other entities configured to learn from or train on input data, and to apply the learning or training to provide results or analysis for subsequent data.

Machine learning-based software may include a model generator, a training data module, a model processor, a model memory, and a communication device. Machine learning-based software may be configured to create prediction models based on the training data. In some embodiments, machine learning-based software may generate decision trees. For example, machine learning-based software may generate nodes, splits, and branches in a decision tree. Machine learning-based software may also calculate coefficients and hyper parameters of a decision tree based on the training data set. In other embodiments, machine learning-based software may use Bayesian algorithms or clustering algorithms to generate predicting models. In yet other embodiments, machine learning-based software may use association rule mining, artificial neural networks, and/or deep learning algorithms to develop models. In some embodiments, to improve the efficiency of the model generation, machine learning-based software may utilize hardware optimized for machine learning functions, such as an FPGA.

The systems and methods may support different hardware platforms/architectures, may add implementations for new network layers and new hardware platforms/architectures, and may be optimized in terms of processing, memory and/or other hardware resources for a specific hardware platform/architecture being targeted. Examples of platforms are different GPUs (e.g., Nvidia GPUs, ARM Mali GPUs, AMD GPUs, etc.), different forms of CPUs (e.g., Intel Xeon, ARM, TI, etc.), and programmable logic devices, such as Field Programmable Gate Arrays (FPGAs).

Exemplary target platforms include host computers having one or more single core and/or multicore CPUs and one or more Parallel Processing Units (PPUs), such as Graphics Processing Units (GPUs), and embedded systems including single and/or multicore CPUs, microprocessors, Digital Signal Processors (DSPs), and/or Field Programmable Gate Arrays (FPGAs).

The subject matter described herein may be executed using a distributed computing environment. The environment may include client and server devices, interconnected by one or more networks. The distributed computing environment also may include target platforms. The target platform may include a multicore processor. Target platform may include a host (Central Processing Unit) and a device (Graphics Processing Unit). The servers may include applications or processes accessible by the clients. The devices of the environment may interconnect via wired connections, wireless connections, or a combination of wired and wireless connections.

The servers may include one or more devices capable of receiving, generating, storing, processing, executing, and/or providing information. For example, servers may include a computing device, such as a server, a desktop computer, a laptop computer, a tablet computer, a handheld computer, or a similar device.

The clients may be capable of receiving, generating, storing, processing, executing, and/or providing information. Information may include any type of machine-readable information having substantially any format that may be adapted for use, e.g., in one or more networks and/or with one or more devices. The information may include digital information and/or analog information. The information may further be packetized and/or non-packetized. In an embodiment, the clients may download data and/or code from the servers via the network. In some implementations, the clients may be desktop computers, workstations, laptop computers, tablet computers, handheld computers, mobile phones (e.g., smart phones, radiotelephones, etc.), electronic readers, or similar devices. In some implementations, the clients may receive information from and/or transmit information to the servers.

The subject matter described herein and/or one or more of its parts or components may comprise registers and combinational logic configured and arranged to produce sequential logic circuits. In some embodiments, the subject matter described herein may be implemented through one or more software modules or libraries containing program instructions pertaining to the methods described herein, that may be stored in memory and/or on computer readable media, and may be executed by one or more processors. Other computer readable media may also be used to store and execute these program instructions. In alternative embodiments, various combinations of software and hardware, including firmware, may be utilized to implement the present disclosure.

A person having ordinary skill in the art will recognize that the principles described herein may be applied to other physical systems not explicitly described herein, as the model described herein here provides a framework that is not specific to any particular physical system but rather can be used to build surrogates that represent components of any physical system.

The descriptions of the various embodiments of the technology disclosed herein have been presented for purposes of illustration, but these descriptions are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system having a multiple-data fitting interface for design optimization, the system having at least one processor configured for:
receiving data objects that include pairs of data with information in the form of one or more computational models that model the behavior of multiple items in a system;
pairing the received data objects into a collection, wherein the collection includes dependencies between the received data objects;
generating a virtual population based on the received data objects, wherein the virtual population comprises multiple virtual items, with each virtual item comprising a combination of model parameters that describe data in the received data objects;
generating a prediction for the virtual population using a global sensitivity analysis across the virtual population, wherein the prediction includes a determined cost and an optimal configuration for the virtual population; and
generating an output, wherein the output includes a visualization of the prediction for the virtual population.

2. The system of claim 1, wherein the computational models include a differential equation.

3. The system of claim 1, wherein the computational models include a stochastic model.

4. The system of claim 1, wherein the parameters for the virtual population are optimized simultaneously.

5. The system of claim 1, wherein the parameters for the virtual population are optimized sequentially.

6. The system of claim 1, wherein the received data objects include clinical trial data, and wherein the virtual items include virtual patients in a clinical trial.

7. The system of claim 1, wherein the computational models are defined using a common interexchange format.

8. The system of claim 1, wherein the computational models are defined using a symbolic domain-specific language representation.

9. A method for design optimization using a multiple-data fitting interface, the method comprising:
receiving data objects that include pairs of data with information in the form of one or more computational models that model the behavior of multiple items in a system;
pairing the received data objects into a collection, wherein the collection includes dependencies between the received data objects;
generating a virtual population based on the received data objects, wherein the virtual population comprises multiple virtual items, with each virtual item comprising a combination of model parameters that describe data in the received data objects;
generating a prediction for the virtual population using a global sensitivity analysis across the virtual population, wherein the prediction includes a determined cost and an optimal configuration for the virtual population; and
generating an output, wherein the output includes a visualization of the prediction for the virtual population.

10. The method of claim 9, wherein the computational models include a differential equation.

11. The method of claim 9, wherein the computational models include a stochastic model.

12. The method of claim 9, wherein the parameters for the virtual population are optimized simultaneously.

13. The method of claim 9, wherein the parameters for the virtual population are optimized sequentially.

14. The system of claim 9, wherein the received data objects include clinical trial data, and wherein the virtual items include virtual patients in a clinical trial.

15. The system of claim 9, wherein the computational models are defined using a common interexchange format or are defined using a symbolic domain-specific language representation.

16. A system having a multiple-data fitting interface for design optimization, the system having at least one processor configured for:
receiving data objects that include pairs of data with information in the form of one or more computational models that model the behavior of multiple patients in a clinical trial;
pairing the received data objects into a collection that includes dependencies between the received data objects;
generating a virtual population based on the received data objects,
wherein the virtual population comprises multiple virtual patients, with each virtual patient comprising a combination of model parameters that describe data in the received data objects, and
wherein the generation of the virtual population includes a user-determined or default cost function and an algorithm for finding an optimal configuration for the virtual population with respect to said cost;
generating a prediction for the virtual population by simulating with each of the virtual patients; and
generating an output, wherein the output includes a visualization of the prediction for the virtual population.

17. The system of claim 16, wherein the computational models are defined using a symbolic domain-specific language representation.

18. The system of claim 16, wherein the different trial types include a normal trial and a steady state trial.

19. The system of claim 16, wherein the prediction is based on a constant rate added to a differential equation for a fixed duration.

20. The system of claim 16, wherein each calibrated set of parameters is a virtual patient, and a virtual population is a collection of virtual patients.
